(19) United States
(12) Patent Application Publication
(10) Pub. No.: US 2026/0209777 A1
(43) Pub. Date: Jul. 23, 2026

(54) TARGETING GLYCOSYLPHOSPHATIDYLINOSITOL (GPI) PATHWAY PROTEINS TO TREAT OVARIAN CANCER

(71) Applicant: UNIVERSITY OF MASSACHUSETTS, Westborough, MA (US)

(72) Inventors: Michael R. GREEN, Boylston, MA (US); Alok K. MISHRA, Worcester, MA (US); Sunil K. MALONIA, Worcester, MA (US)

(21) Appl. No.: 19/143,103

(22) PCT Filed: Apr. 17, 2024
(86) PCT No.: PCT/US2024/024946

§ 371 Date: Jun. 25, 2025

Related U.S. Application Data
(60) Provisional application No. 63/460,163, filed on Apr. 18, 2023.

(51) Int. Cl. C12N 15/113; A61K 31/198; A61K 31/216; A61K 31/426; A61K 31/7105; A61K 31/713; A61K 45/06; A61P 35/00

(52) U.S. Cl. CPC C12N 15/1138; A61K 31/198; A61K 31/216; A61K 31/426; A61K 31/7105; A61K 31/713; A61K 45/06; A61P 35/00; C12N 2310/141

(57) ABSTRACT

Methods for treating an ovarian cancer involving inhibition of a glycosylphosphatidylinositol (GPI) pathway protein such as glycosylphosphatidylinositol anchor attachment 1 (GPAA1), phosphatidylinositol glycan anchor biosynthesis class K (PIGK), phosphatidylinositol glycan anchor biosynthesis class P (PIGP), phosphatidylinositol glycan anchor biosynthesis class T (PIGT), and phosphatidylinositol glycan anchor biosynthesis class U protein (PIGU).

### CLAIM OF PRIORITY

This application claims the benefit of U.S. Provisional Patent Application No. 63/460,163, filed on Apr. 18, 2023, which is incorporated by reference herein in its entirety.

### SEQUENCE LISTING

This application contains a Sequence Listing that has been submitted electronically as an XML file named 07917-0444WO1_ST26_SL.xml. The XML file, created on Apr. 17, 2024, is 19,542 bytes in size. The material in the XML file is hereby incorporated by reference in its entirety.

### BACKGROUND OF THE INVENTION

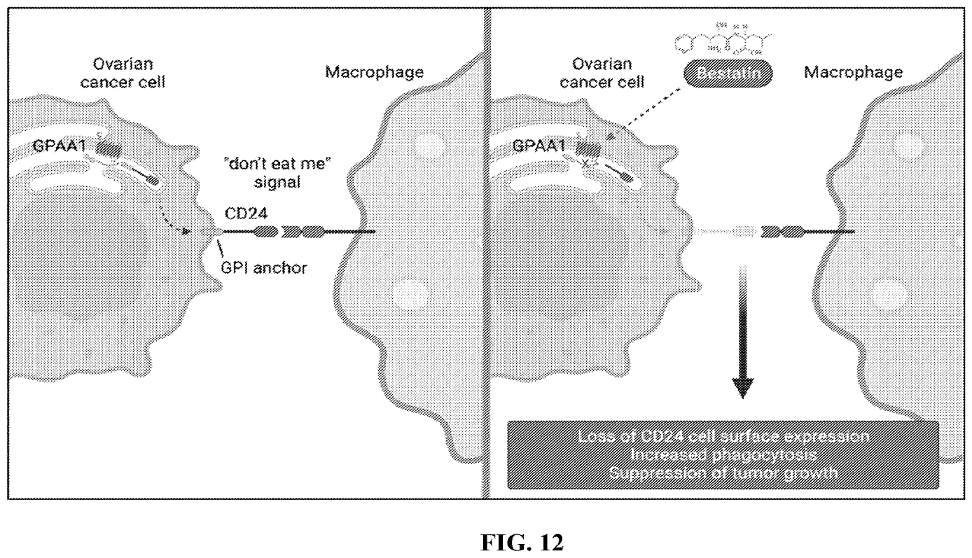

Ovarian cancer is the second most common gynecologic cancer, and the leading cause of death from gynecologic malignancies in the United States (American Cancer Society, Cancer Facts & FIGS. 2022). The most common type of ovarian cancer is epithelial ovarian cancer, which accounts for approximately 90% of ovarian tumors. Epithelial ovarian cancer is categorized into different types, with high-grade serous ovarian carcinoma (HGSOC) being the most common type of invasive epithelial ovarian cancer, accounting for approximately 75% of epithelial ovarian cancers. HGSOC is an aggressive cancer that does not present with early symptoms, and thus the majority of HGSOC patients are diagnosed at an advanced stage and have a poor prognosis, with a 5-year survival rate of about 30%.

### SUMMARY OF THE INVENTION

The present disclosure is based, at least in part, on the novel and unexpected findings that genetic or pharmacological inhibition of the GPI pathway protein GPAA1 suppressed growth of tumors derived from human ovarian cancer cells in xenograft mice.

Accordingly, aspects of the present disclosure provide a method of treating ovarian cancer, the method comprising administering to a subject in need thereof an effective amount of an inhibitor of a glycosylphosphatidylinositol (GPI) pathway protein.

In some embodiments, the GPI pathway protein comprises glycosylphosphatidylinositol anchor attachment 1 (GPAA1), phosphatidylinositol glycan anchor biosynthesis class P (PIGK), phosphatidylinositol glycan anchor biosynthesis class P (PIGP), phosphatidylinositol glycan anchor biosynthesis class T (PIGT), or phosphatidylinositol glycan anchor biosynthesis class U protein (PIGU).

In some embodiments, the inhibitor comprises a small molecule inhibitor, a peptide inhibitor, an antibody or antigen binding fragment thereof, an agent that inhibits expression of the GPI pathway protein, or a combination thereof.

In some embodiments, the small molecule inhibitor comprises a metallo-aminopeptidase inhibitor. In some embodiments, the metallo-aminopeptidase inhibitor comprises bestatin, LYP, LYP3, tosedostat, ARM1, or a combination thereof.

In some embodiments, the agent that inhibits expression of the GPI pathway protein comprises a short interfering nucleic acid (siNA), a short interfering RNA (siRNA), a double-stranded RNA (dsRNA), a micro-RNA (miRNA), a short hairpin RNA (shRNA), or a combination thereof.

In some embodiments, the inhibitor is formulated in a pharmaceutical composition, which further comprises a pharmaceutically acceptable carrier.

In some embodiments, the subject is a human subject having or at risk for having ovarian cancer. In some embodiments, the ovarian cancer comprises epithelial ovarian cancer, germ cell ovarian cancer, stromal cell ovarian cancer, small cell carcinoma (SCCO), or combinations thereof. In some embodiments, the ovarian cancer comprises chemo-resistant ovarian cancer.

In some embodiments, ovarian cancer cells in a biological sample obtained from the human subject express CD24. In some embodiments, ovarian cancer cells in a biological sample obtained from the human subject express GPAA1. In some embodiments, ovarian cancer cells in a biological sample obtained from the human subject express PEPT1 and/or PEPT2.

In some embodiments, methods described herein further comprise administering to the subject an additional anti-cancer therapy.

### DETAILED DESCRIPTION OF THE DRAWINGS

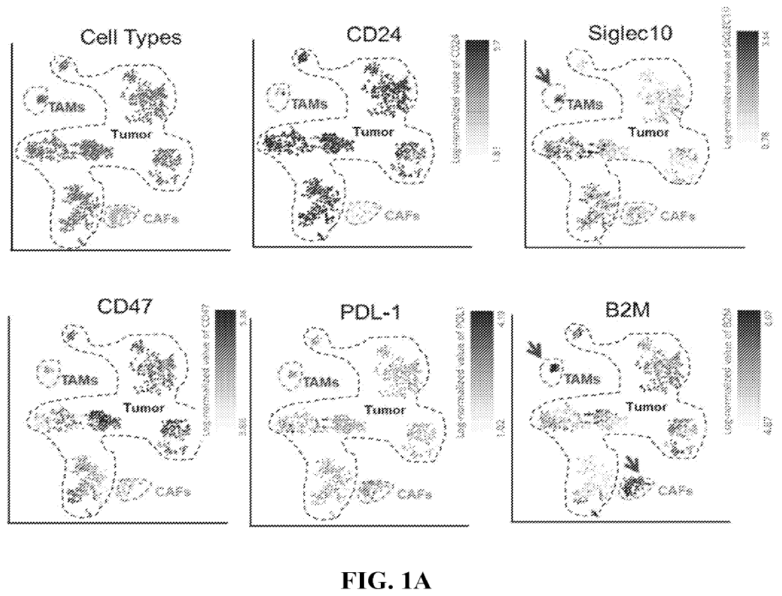

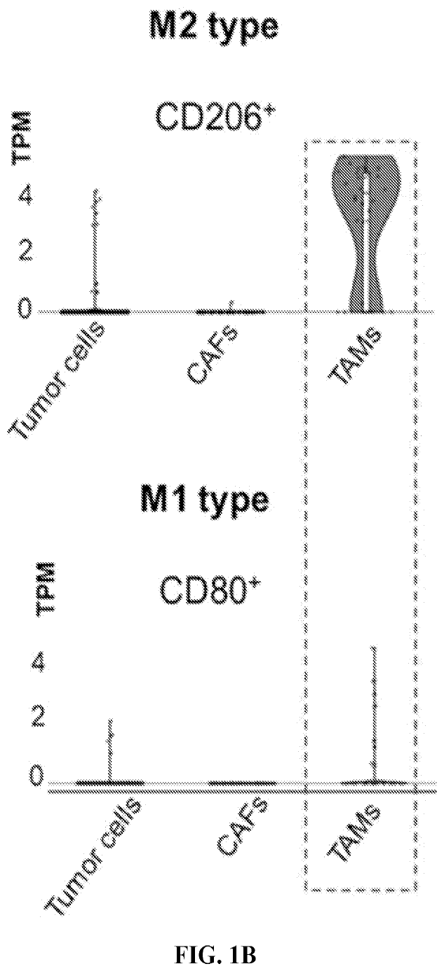

FIGS. 1A-1B: Single-cell analysis and expression of innate immune components in ovarian cancer. (FIG. 1A) Uniform manifold approximation and projection (UMAP) dimension 1 and 2 plots displaying ~11,000 single ovarian cancer cells obtained from 22 ascites specimens from 11 patients with HGSOC (source data from GSE146026; Izar et al., Nat Med 2020, 26(8):1271-1279). In the upper left panel, cells are colored by cluster identity. The remaining five panels show CD24, Siglec10, CD47, PDL-1 and B2M expression overlaid onto UMAP space. The results show that CD24 is highly expressed in ovarian tumor cells with weak expression in tumor-associated macrophages (TAMs) and cancer-associated fibroblasts (CAFs). By contrast, CD47 is expressed in both cancer and non-cancer cells, PDL-1 expression is very low, and B2M expression is higher in TAMs and CAFs than in cancer cells. Collectively, these observations suggest that CD24 is a more tumor specific immune checkpoint in ovarian cancer compared to other innate immune checkpoints. Moreover, Siglec10 is expressed primarily in TAMs. The plots were generated using the BioTuring Browser. (FIG. 1B) Violin plots showing expression of CD206 (an M2 macrophage marker) and CD80 (an M1 macrophage marker) in ovarian tumor cells, TAMs and CAFs. The results show that in ovarian cancer, the TAMs are predominantly M2 type.

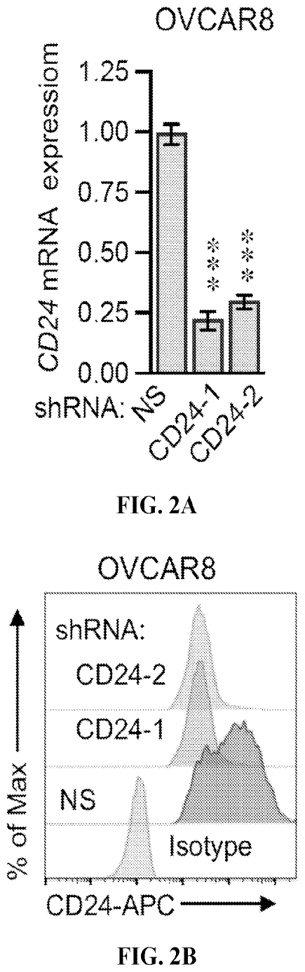

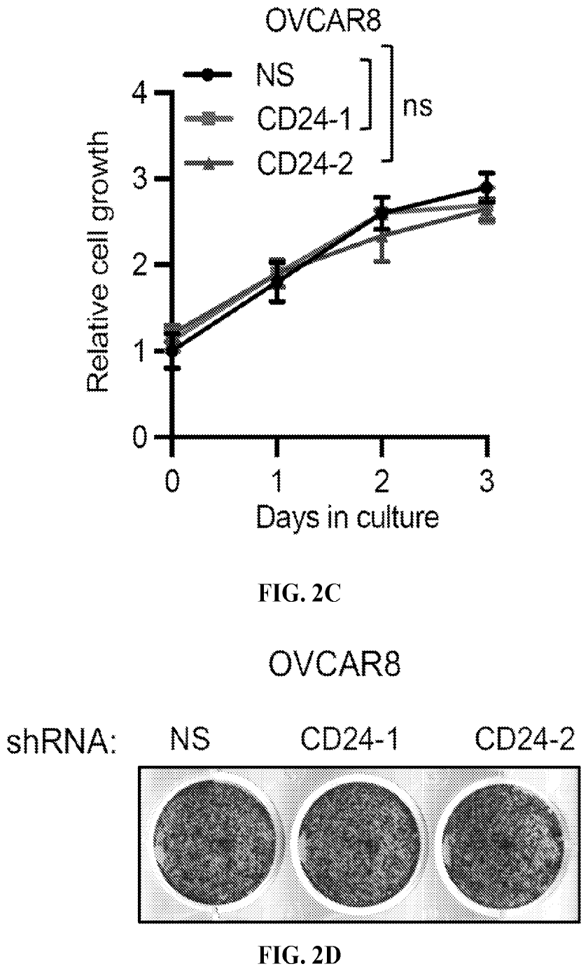

FIGS. 2A-2D: CD24 is not required for viability of OVCAR8 human ovarian cancer cells. (FIG. 2A) qRT-PCR analysis monitoring knockdown efficiency of two independent shRNAs targeting CD24. CD24 expression in OVCAR8 cells was normalized to that obtained with a control non-silencing (NS) shRNA, which was set to 1. Error bars indicate SD (n=3 technical replicates of a representative experiment (out of two independent experiments). P values were calculated using one-way ANOVA followed by Dunnett's multiple comparisons test. ***P<0.001. (FIG. 2B) Flow cytometry analysis monitoring cell surface expression of CD24 in OVCAR8 cells expressing a NS shRNA or one of two independent CD24 shRNAs. As a control, isotype IgG antibody was used. (FIG. 2C) Cell viability assay. OVCAR8 cells expressing NS or CD24 shRNA were cultured for indicated times and analyzed for cell viability by PrestoBlue assay. Error bars indicate SD (n=3 technical replicates of a representative experiment (out of two independent experiments)). P values were calculated using one-way ANOVA, and the differences were not significant (ns). (FIG. 2D) Crystal violet staining. 1×10⁴ OVCAR8 cells expressing NS or CD24 shRNA were seeded in each well and cultured for 5 days, followed by crystal violet staining.

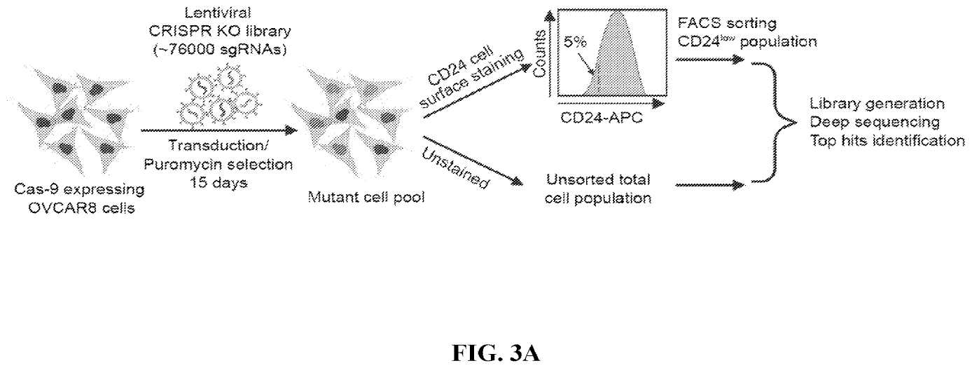

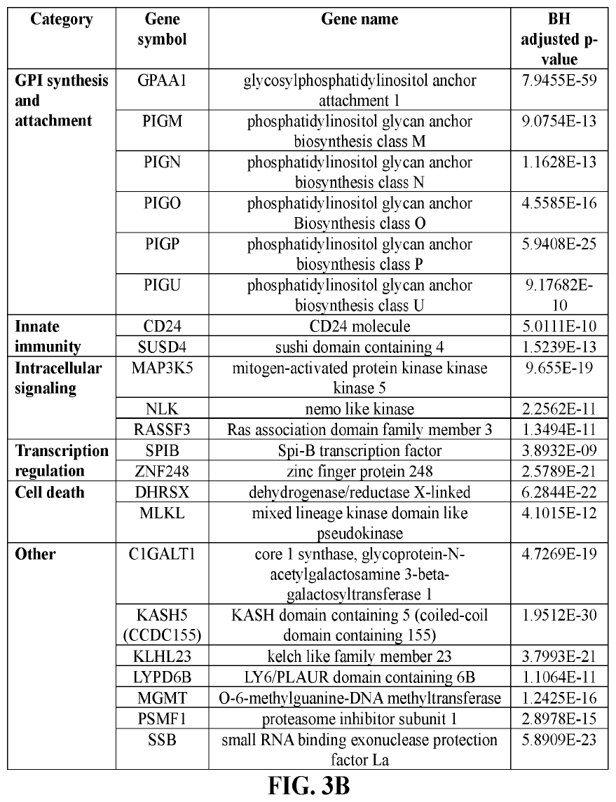

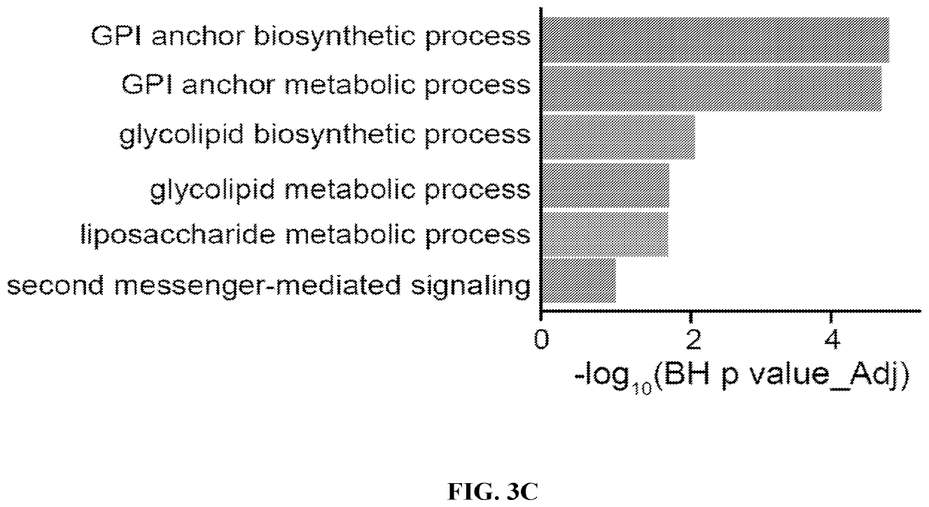

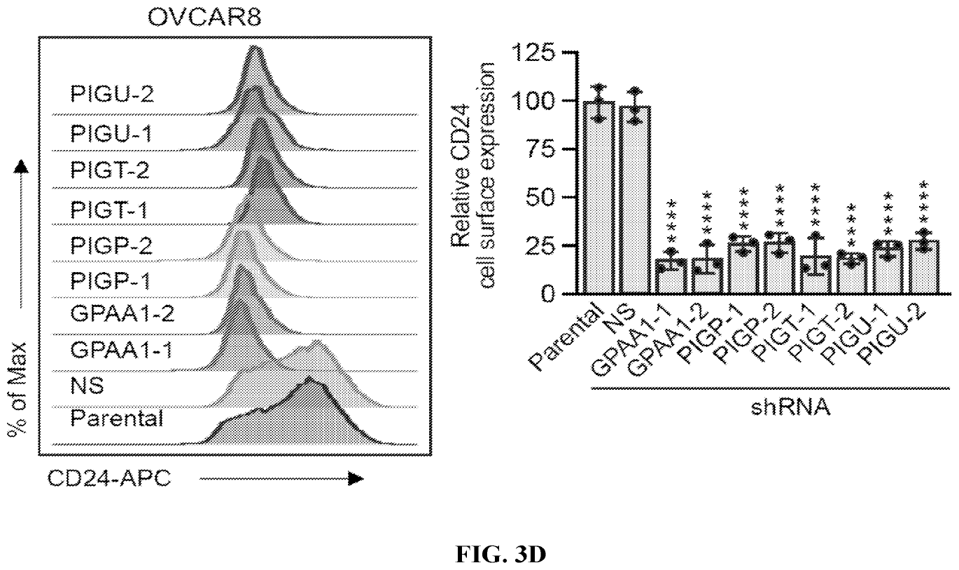

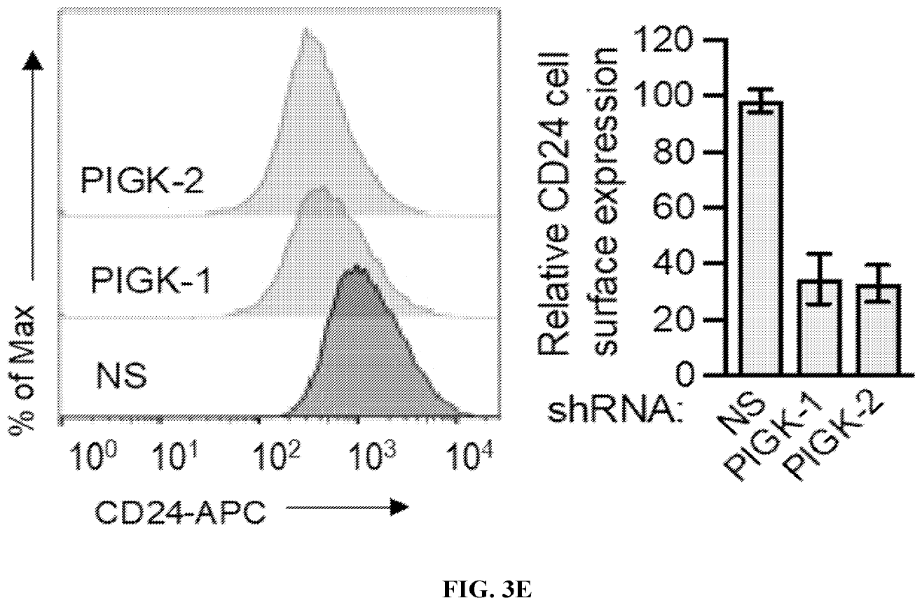

FIGS. 3A-3E: A genome-wide CRISPR/Cas9 screen identifies factors that promote cell surface expression of CD24 in ovarian cancer. (FIG. 3A) Schematic of the CRISPR/Cas9 screening strategy. (FIG. 3B) Table showing candidate genes with at least three significantly enriched sgRNAs, of which at least two sgRNAs were among the top 10% most significant sgRNA. (FIG. 3C) GO analysis showing that the most significant biological processes related to the enriched sgRNAs. (FIG. 3D) Validation of representative candidates involved in GPI synthesis and attachment by CD24 flow cytometry analysis. (Left), Representative histograms for parental OVCAR8-Cas9 cells expressing two independent shRNAs targeting GPAA1, PIGP, PIGT or PIGU, or as a control a non-silencing (NS) shRNA. (Right), Quantification of mean fluorescence intensity (MFI). The results were normalized to that obtained in OVCAR8-Cas9 cells, which was set to 100% MFI. Error bars indicate SD (n=3 technical replicates of a representative experiment (out of two independent experiments). (FIG. 3E) (Left), Representative histograms for OVCAR8 cells expressing two independent shRNAs targeting PIGK, or as a control a non-silencing (NS) shRNA. (Right), Quantification of mean fluorescence intensity (MFI). The results were normalized to that obtained in OVCAR8 expressing NS, which was set to 100% MFI. Error bars indicate SD (n=3 technical replicates of a representative experiment (out of two independent experiments). P values were calculated using one-way ANOVA followed by Dunnett's multiple comparisons test. ****P<0.0001.

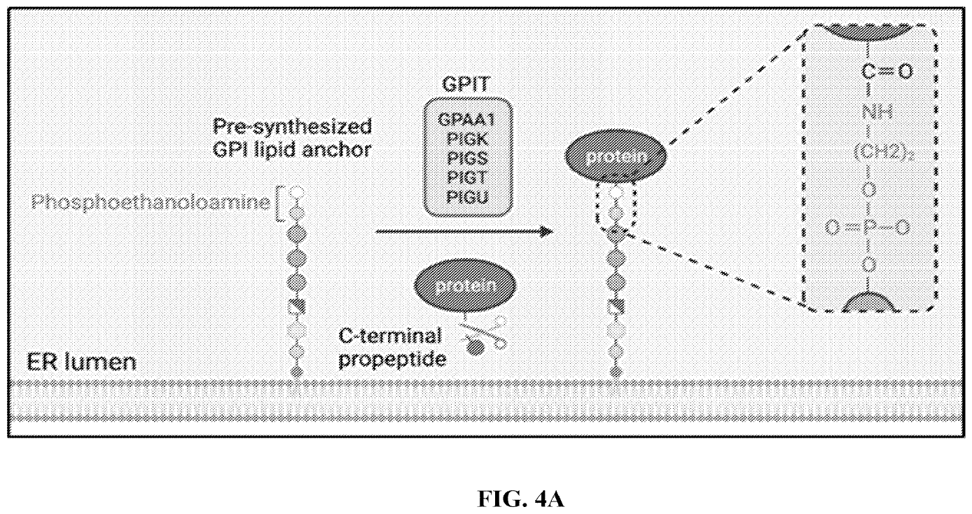

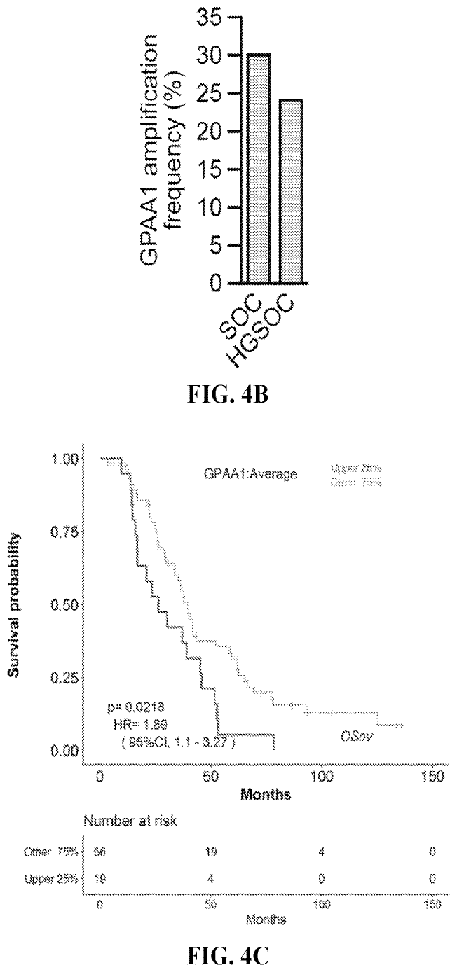

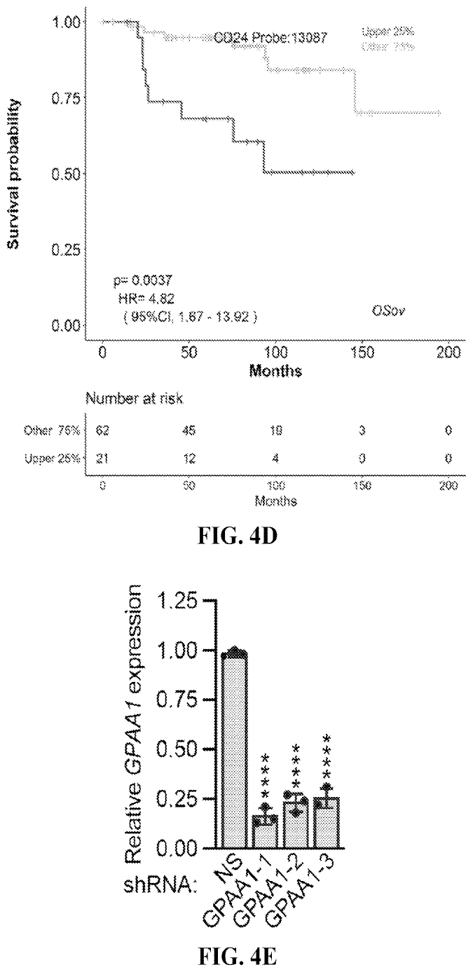

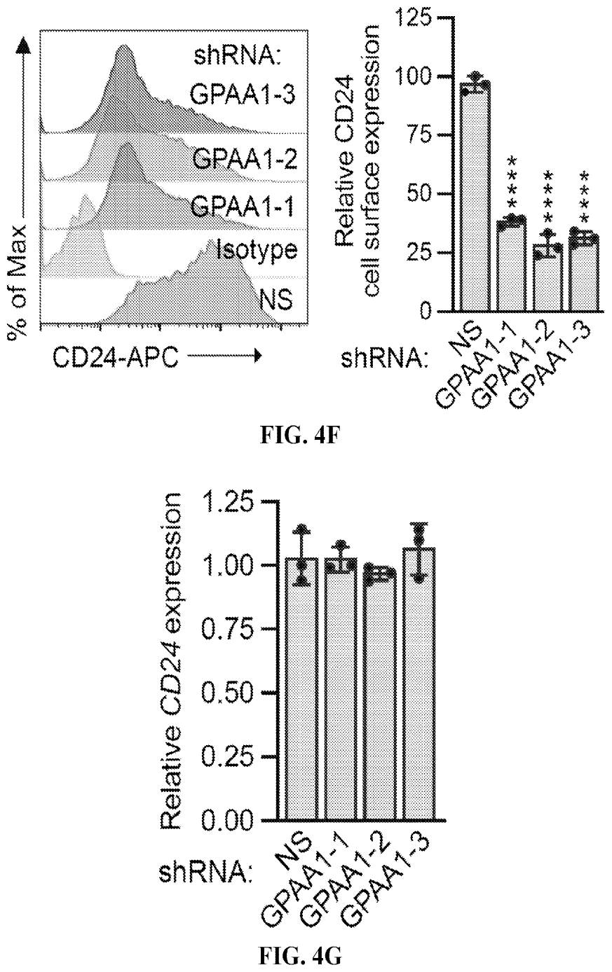

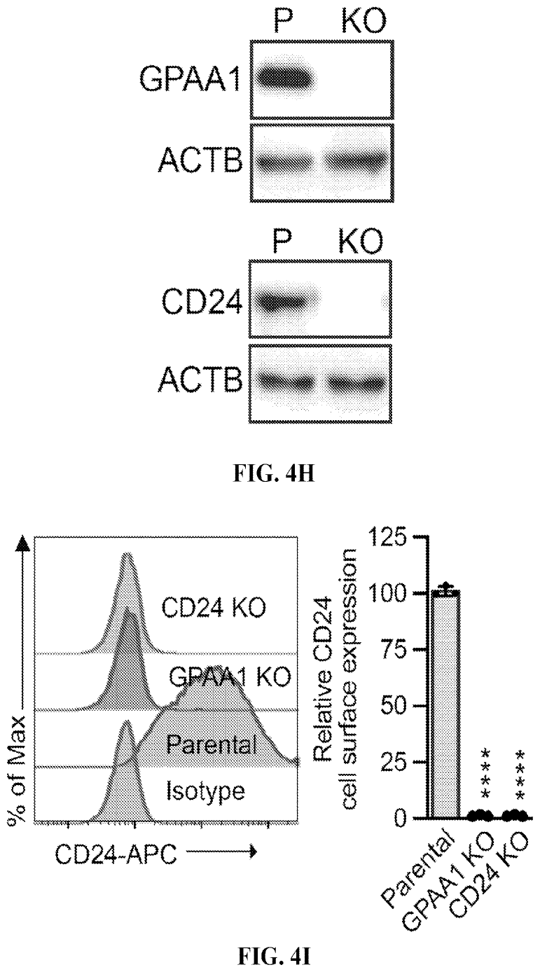

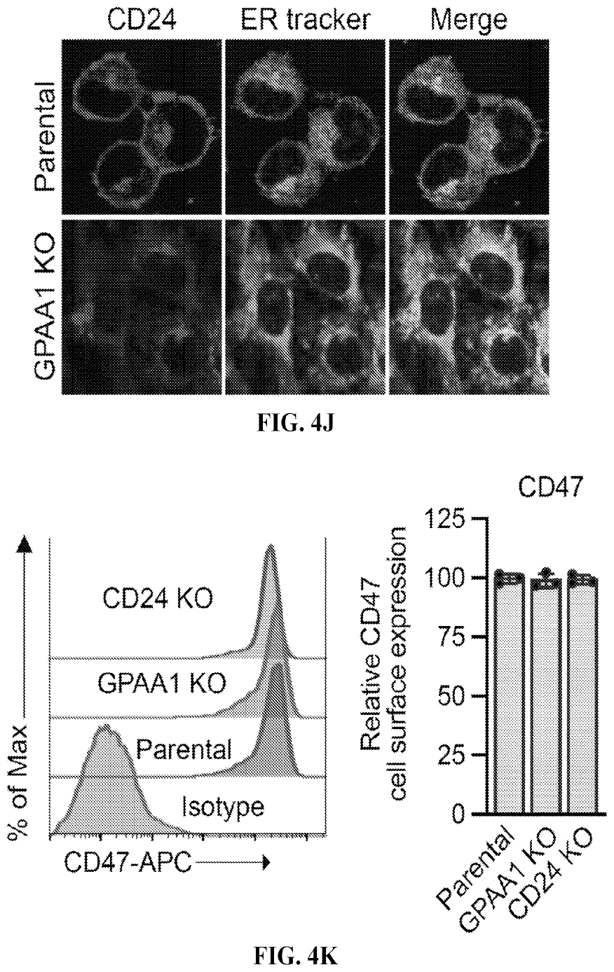

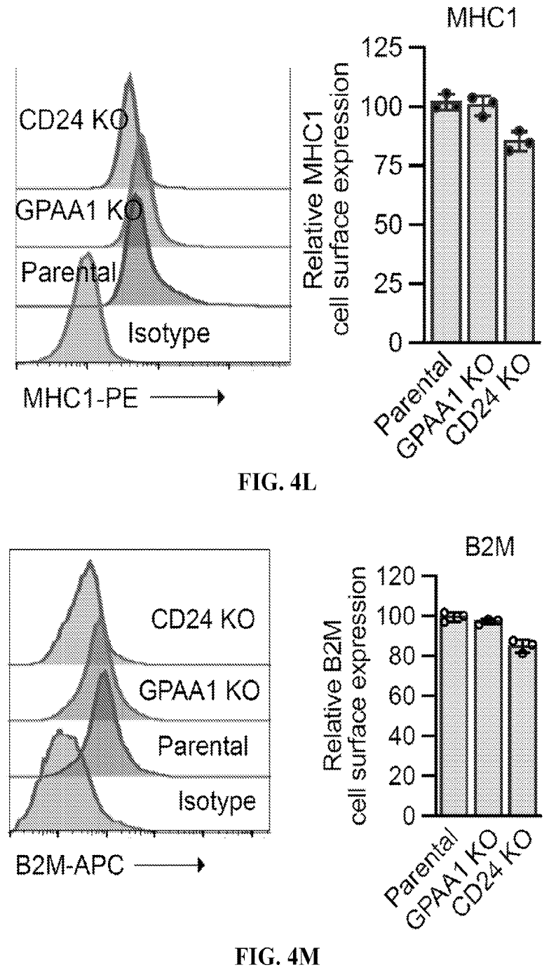

FIGS. 4A-4M: Validation of GPAA1 as a factor that promotes cell surface expression of CD24 in ovarian cancer. (FIG. 4A) Schematic of the GPI attachment pathway, indicating the role for the GPIT complex. The attachment of the protein to phosphoethanolamine is enlarged to show the resemblance to a peptide bond. (FIG. 4B) Bar graph showing the percentage of tumors with GPAA1 amplification in serous ovarian cancer (SOC) and high-grade serous ovarian cancer (HGSOC), source data were obtained through the cBioPortal webservice. (FIGS. 4C-4D) Kaplan-Meier survival analysis showing the correlation between GPAA1 (FIG. 4C) and CD24 (FIG. 4D) expression and overall survival (source data from Lisowska et al., Front Oncol 2014, 4:6). (FIG. 4E) qRT-PCR analysis monitoring knockdown efficiency in OVCAR8 cells of three independent shRNAs targeting GPAA1. The results were normalized to that obtained with a NS shRNA, which was set to 1. Error bars indicate SEM (n=3 technical replicates of a representative experiment (out of two independent experiments)). (FIG. 4F) CD24 flow cytometry analysis in OVCAR8 cells expressing an NS or one of three independent GPAA1 shRNAs. (Left), Representative histograms; (Right), Quantification of MFI. Error bars indicate SD (n=3 biological replicates). (FIG. 4G) qRT-PCR analysis monitoring CD24 expression in OVCAR8 cells expressing NS or one of three independent GPAA1 shRNAs. Error bars indicate SD (n=3 technical replicates of a representative experiment (out of three independent experiments)). (FIG. 4H) Immunoblot analysis monitoring GPAA1 and CD24 protein levels in OVCAR8 GPAA1 knockout (KO) and CD24 KO cells, respectively. The results confirm the lack of GPAA1 and CD24 protein expression in OVCAR8 GPAA1 KO and CD24 KO cells, respectively. P, parental OVCAR8 cells. beta-actin (ACTB) was used as a loading control. (FIG. 4I) CD24 flow cytometry analysis in independently derived GPAA1 KO and CD24 KO OVCAR8 single-cell clones. (Left), Representative histograms; (Right), Quantification of MFI. Error bars indicate SD (n=3 biological replicates). (FIG. 4J) Representative confocal microscopy images of parental OVCAR8 or OVCAR8 GPAA1 KO cells stained with an anti-CD24 antibody or ER tracker. Merged images are shown. (FIGS. 4K-4M) (Left), Flow cytometry analysis of cell surface expression; (Right), Quantification of MFI of non-GPI-linked proteins, CD47 (FIG. 4K), MHC-1 (FIG. 4L) and B2M (FIG. 4M) in parental, GPAA1 KO, and CD24 KO OVCAR8 cells. Error bars indicate SD (n=3 biological replicates). P values were calculated using one-way ANOVA followed by Dunnett's multiple comparisons test. ****P<0.0001.

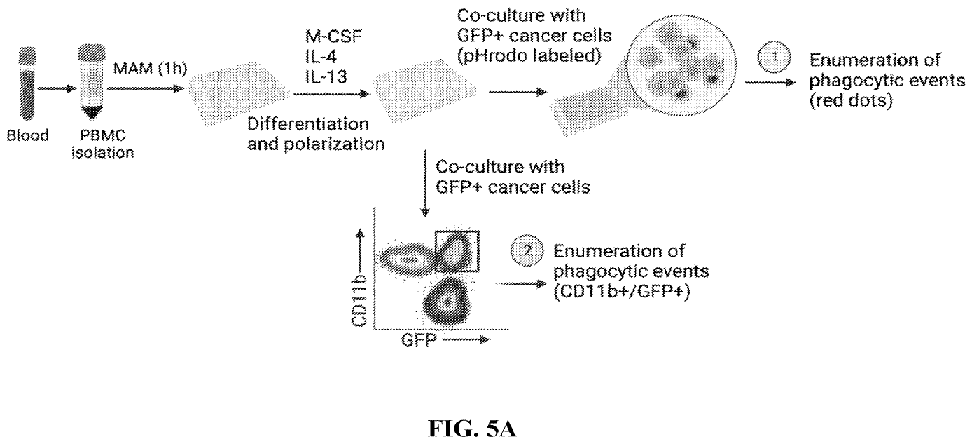

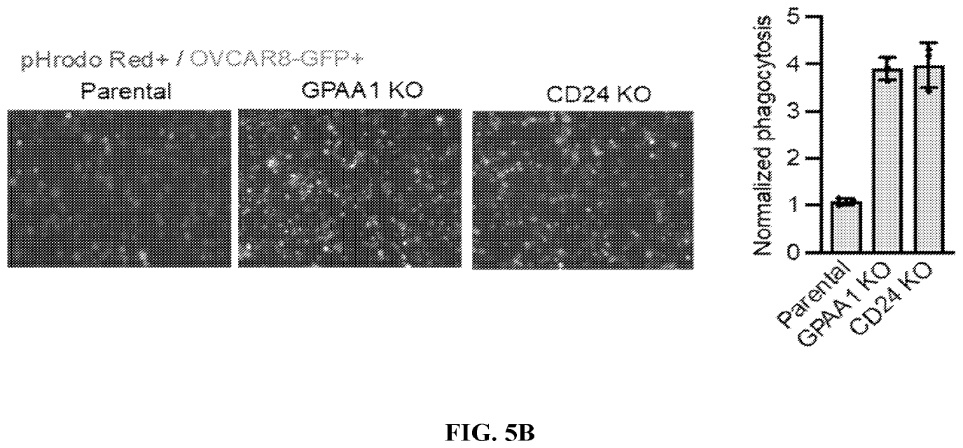

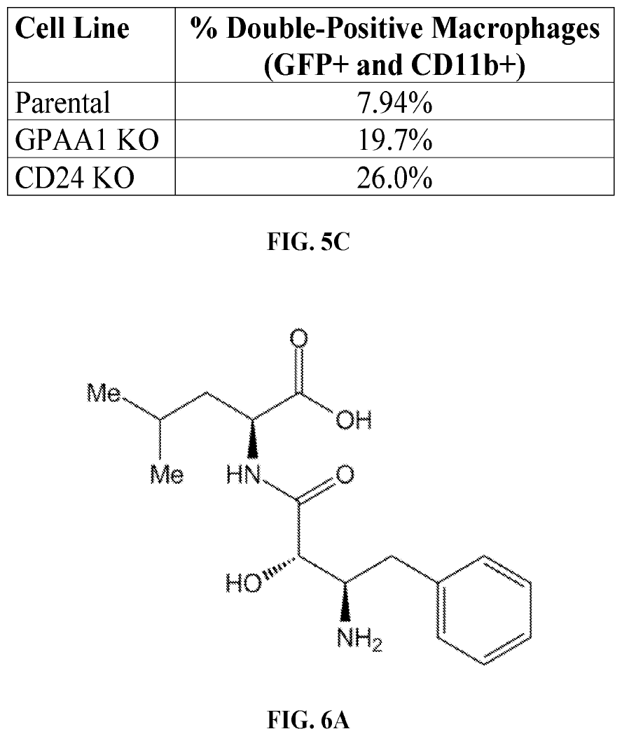

FIGS. 5A-5C: Genetic knockout of GPAA1 promote phagocytosis of ovarian cancer cells by human PBMC-derived macrophages. (FIG. 5A) Schematic of macrophage preparation from human PBMCs and phagocytosis assay. (FIG. 5B) (Left), Representative images; (Right), Quantification of phagocytosis of parental, GPAA1 KO and CD24 KO OVCAR8-GFP cells. The results were normalized to that obtained in parental OVCAR8-GFP cells, which was set to 1. Error bars indicate SD (n=3 technical replicates). P values were calculated using one-way ANOVA followed by Dunnett's multiple comparisons test. ***P<0.001. (FIG. 5C) Flow cytometry results showing double-positive macrophages (GFP+ and CD11b+) in parental, GPAA1 KO and CD24 KO OVCAR8 cells.

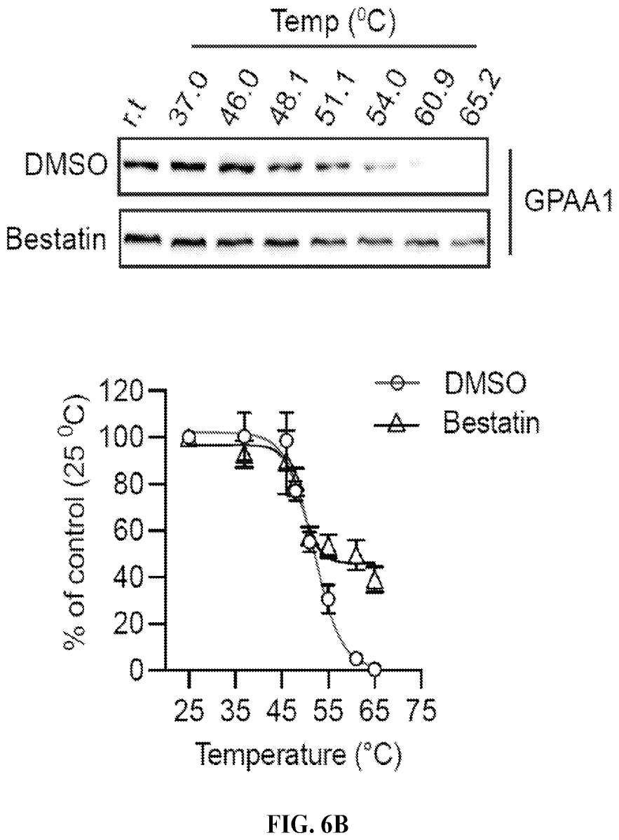

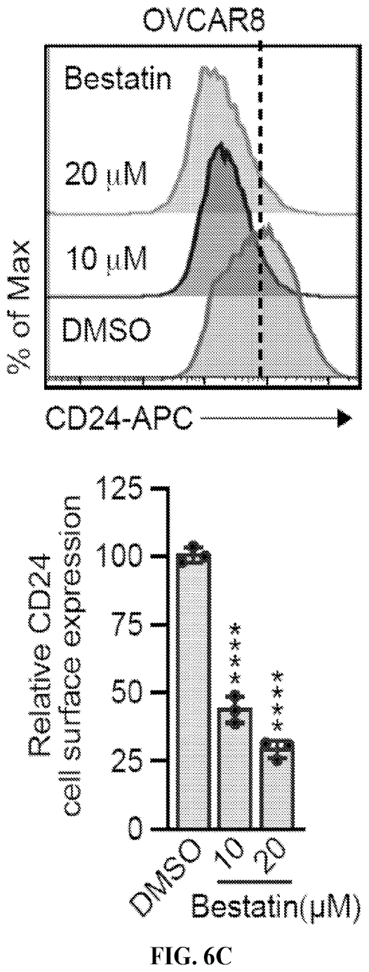

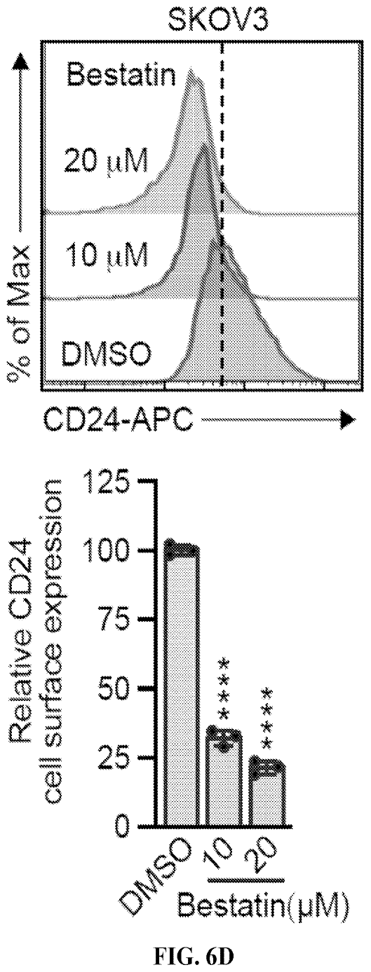

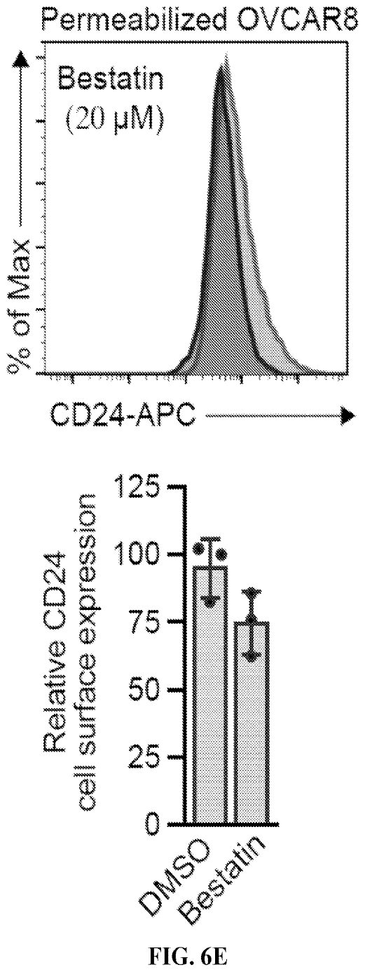

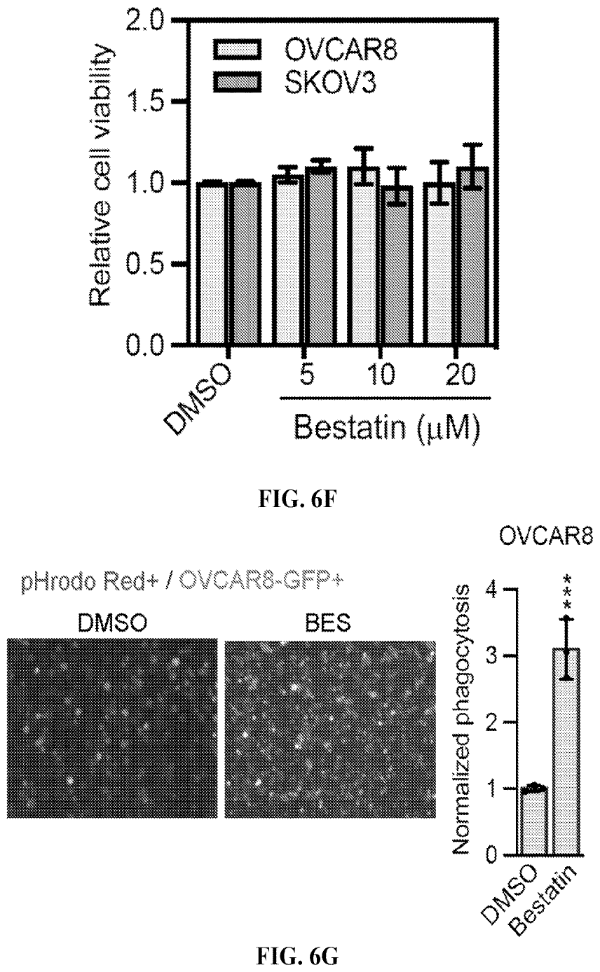

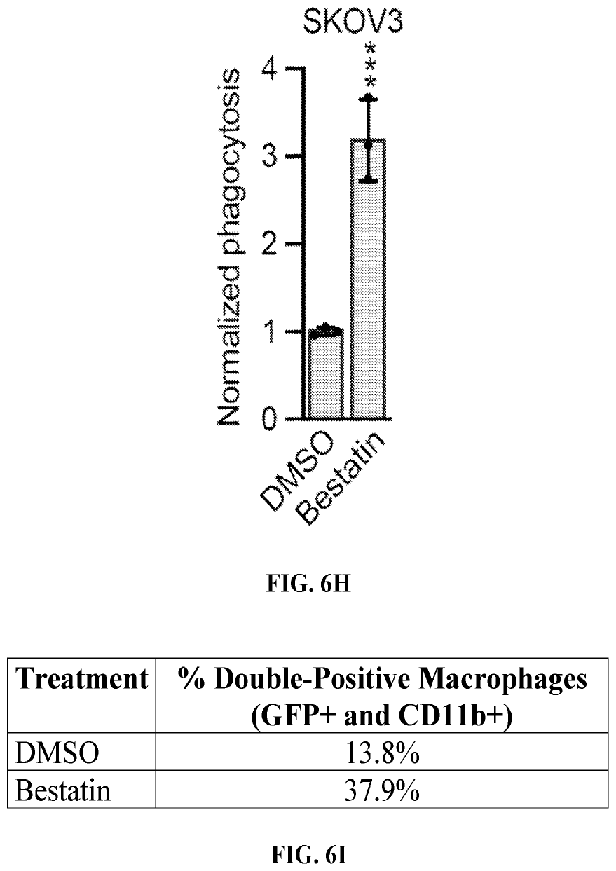

FIGS. 6A-6I: The metallo-aminopeptidase inhibitor bestatin reduces cell surface expression of CD24 and increases phagocytosis of ovarian cancer cells. (FIG. 6A) Chemical structure of bestatin. (FIG. 6B) Cellular thermal stability shift assay (CETSA) showing increased thermal stability of GPAA1 upon binding of bestatin (100 μM) relative to that obtained with vehicle (DMSO). Error bars indicate SD (n=3 biological replicates). (Top), Immunoblot analysis with GPAA1 antibody. (Bottom) The shift in bestatin binding to GPAA1 was analyzed by the Boltzmann sigmoid equation (Graphpad Prism). All data were normalized to the response observed at DMSO-treated conditions at room temperature. (FIGS. 6C-6D) CD24 flow cytometry analysis in OVCAR8 (FIG. 6C) or SKOV3 (FIG. 6D) cells treated with DMSO or 10 or 20 μM bestatin. (Top), Representative histograms; (Bottom), quantification of MFI. Error bars indicate SD (n=3 technical replicates of a representative experiment (out at least three independent experiments)). (FIG. 6E) CD24 flow cytometry analysis in OVCAR8 cells treated with DMSO or 20 μM bestatin and permeabilized using the True-Nuclear Transcription Factor Buffer Set (BioLegend). (Top), Representative histograms; (Bottom), Quantification of MFI. Error bars indicate SD (n=3 technical replicates). (FIG. 6F) Cell viability assay in OVCAR8 and SKOV3 cells treated with DMSO or 5, 10 or 20 μM bestatin. Error bars indicate SD (n=3 technical replicates of a representative experiment (out of two independent experiments). (FIG. 6G) (Left), Representative images; (Right), Quantification of phagocytosis of OVCAR8-GFP cells treated with bestatin (20 μM) or, as a control, DMSO. The results were normalized to that obtained with DMSO, which was set to 1. Error bars indicate SD (n=5 technical replicates). (FIG. 6H) Quantification of phagocytosis of SKOV3 cells treated with bestatin (20 μM) or, as a control, DMSO. Error bars indicate SEM (n=5 technical replicates). (FIG. 6I) Flow cytometry results showing double-positive macrophages (GFP+ and CD11b+) following treatment with DMSO or 20 μM bestatin. P values were calculated using student t test and one-way ANOVA followed by Dunnett's multiple comparisons test, ***P<0.001, ****P<0.0001.

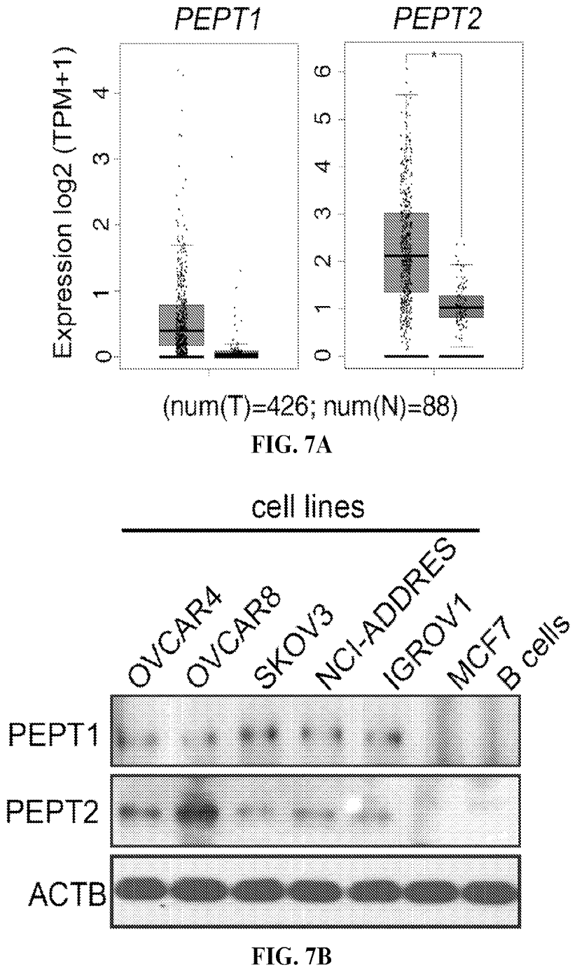

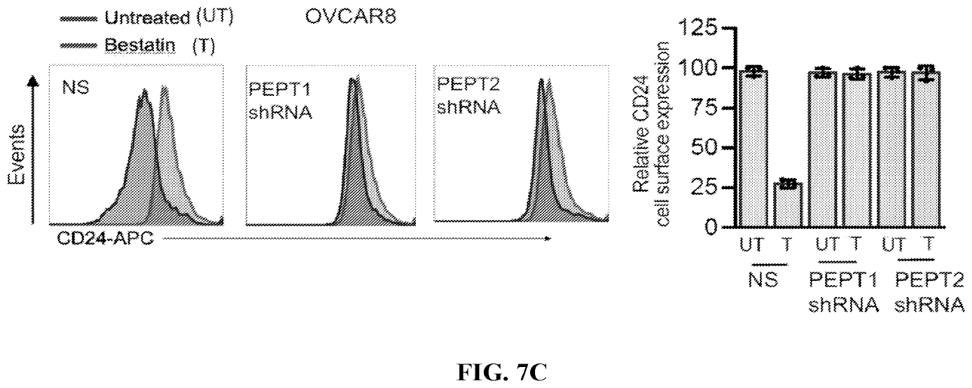

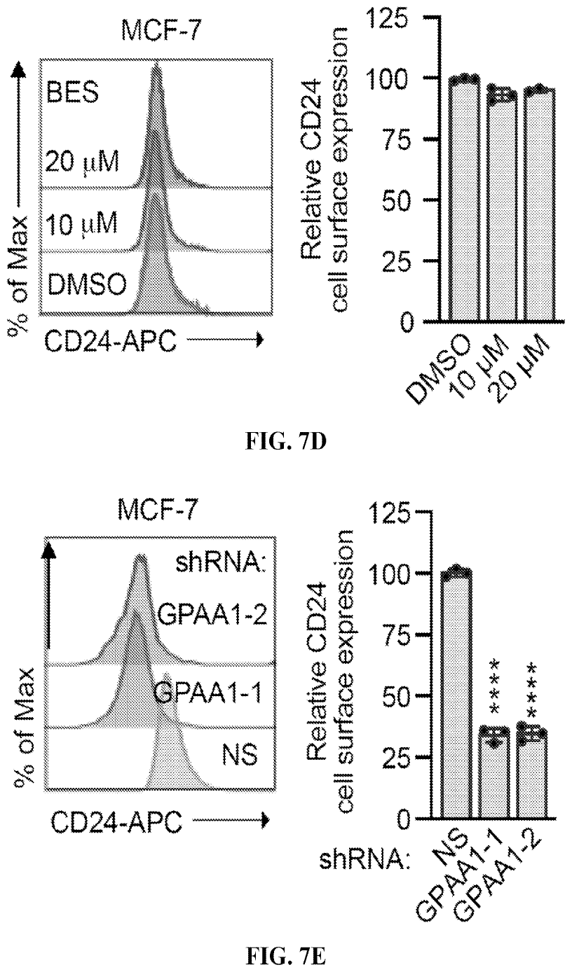

FIGS. 7A-7E: Sensitivity of ovarian cancer cells to bestatin requires expression of the dipeptide transporter PEPT1 or PEPT2. (FIG. 7A) TCGA data showing expression of PEPT1 and PEPT2 in normal ovarian epithelial tissue (left bar) and ovarian cancer tumors (right bar). (FIG. 7B) Immunoblot showing levels of PEPT1 and PEPT2 in several human ovarian cancer cell lines (OVCAR4, OVCAR8, SKOV3, NCI-ADDRES, IGROV1), breast cancer MCF7 cells and primary B cells. ACTB is used as a loading control. (FIG. 7C) (Left), CD24 flow cytometry analysis in OVCAR8 cells expressing an NS, PEPT1 or PEPT2 shRNA, either untreated (UT) or treated (T) with 20 μM bestatin. (Right), Quantification of MFI. (FIGS. 7D-7E) CD24 flow cytometry analysis showing in MCF7 cells treated with DMSO or 10 or 20 μM bestatin (FIG. 7D) or expressing an NS or GPAA1 shRNA (FIG. 7E). (Left) Representative histograms; (Right), Quantification of MFI. Error bars indicate SD (n=3 biological replicates). P values were calculated using one-way ANOVA followed by Dunnett's multiple comparisons test. ****P<0.0001.

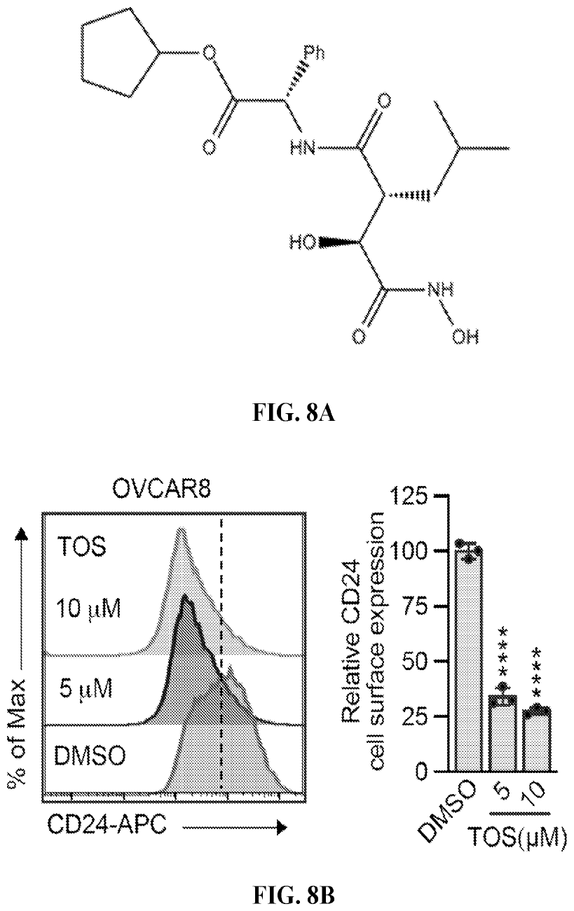

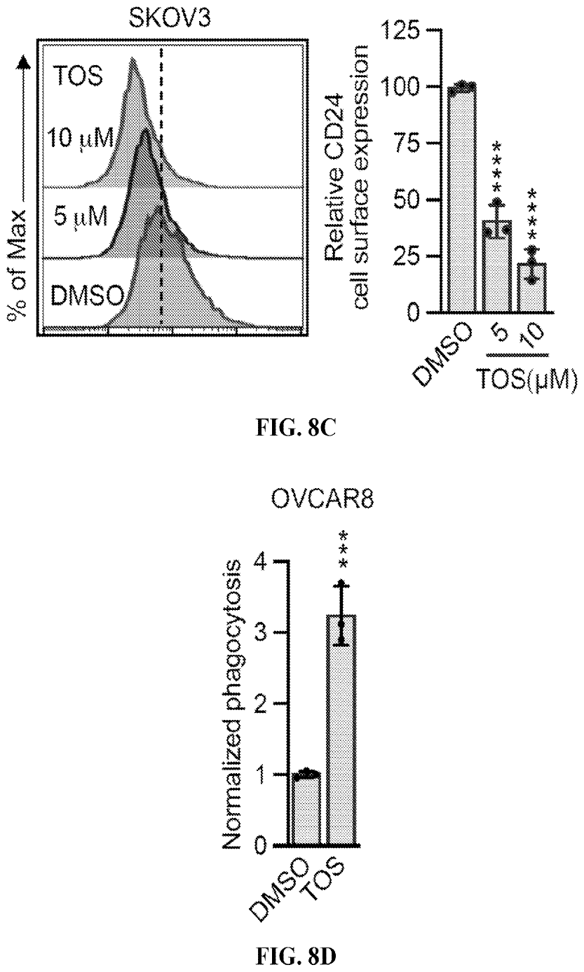

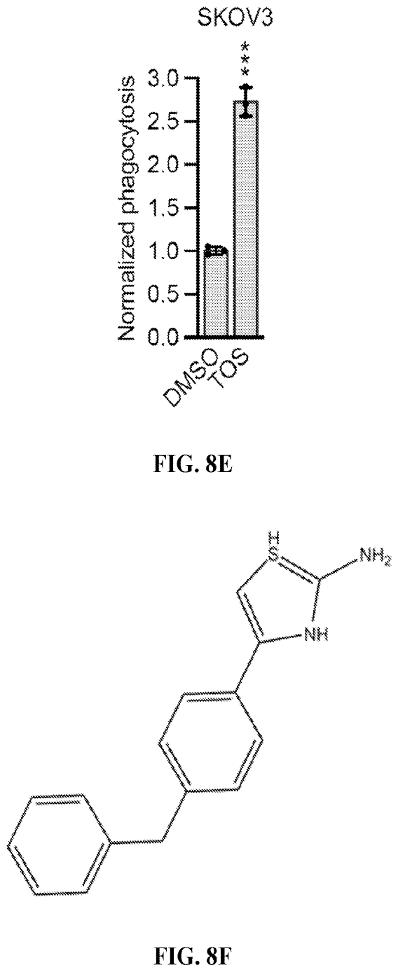

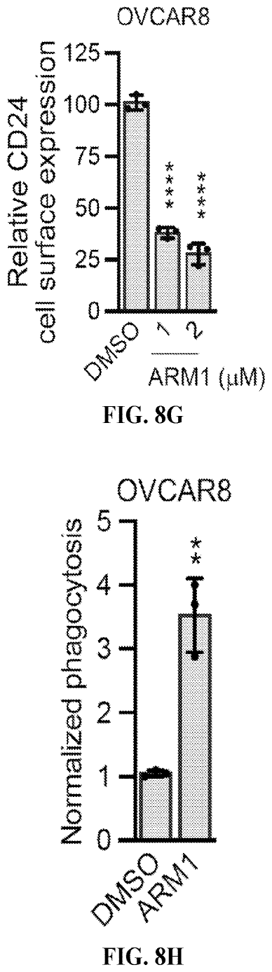

FIGS. 8A-8H: Other metallo-aminopeptidase inhibitors decrease CD24 cell surface expression and increase phagocytosis of ovarian cancer cells. (FIG. 8A) Chemical structure of tosedostat. (FIG. 8B-8C) CD24 flow cytometry analysis in OVCAR8 (FIG. 8B) or SKOV3 (FIG. 8C) cells treated with DMSO or tosedostat (TOS) (5 μM or 10 μM). (Left), Representative histograms; (Right) Quantification of MFI. Error bars indicate SD (n=3 technical replicates). (FIG. 8D-8E) Phagocytosis assay in OVCAR8 (FIG. 8D) and SKOV3 (FIG. 8E) cells treated with DMSO or tosedostat (20 μM). Error bars indicate SD (n=3 technical replicates). (FIG. 8F) Chemical structure of ARM1. (FIG. 8G) CD24 flow cytometry analysis in OVCAR8 cells treated with ARM1 (1 μM or 2 μM). Error bars indicate SD (n=3 technical replicates). (FIG. 8H) Phagocytosis assay in OVCAR8 cells treated with DMSO or ARM1 (2 μM). Error bars indicate SD (n=3 technical replicates). P values were calculated using one-way ANOVA followed by Dunnett's multiple comparisons test. **P<0.01, ***P<0.001, ****P<0.0001.

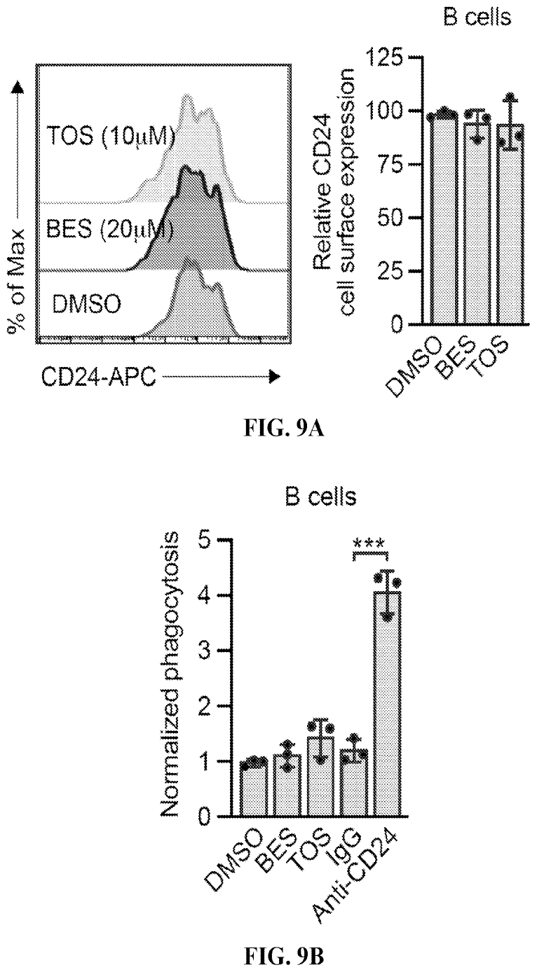

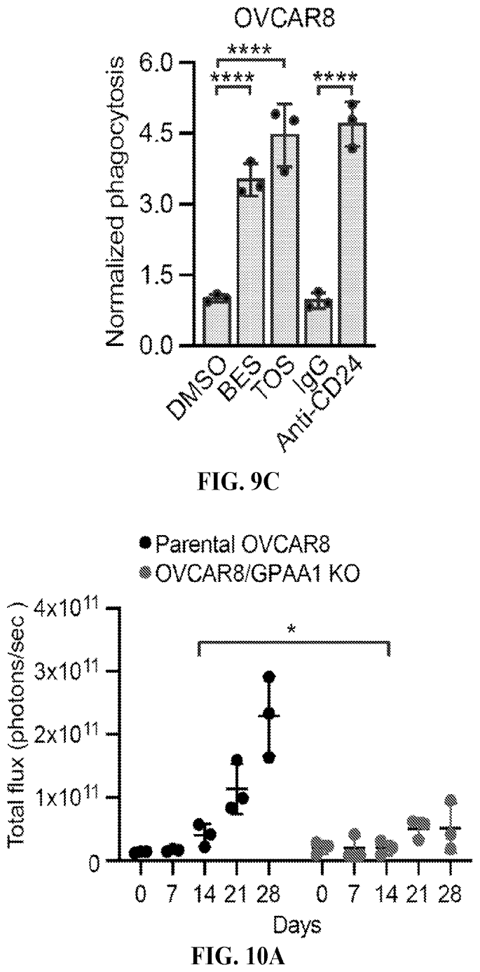

FIGS. 9A-9C: Unlike an anti-CD24 monoclonal antibody, bestatin and tosedostat do not induce phagocytosis of normal B cells. (FIG. 9A) (Left), Representative histogram of flow cytometry analysis showing expression of CD24 on B cells treated with 20 μM bestatin (BES), 10 μM tosedostat (TOS) or as a control, DMSO. (Right) Quantification of MFI. Error bars indicate SD (n=3 technical replicates). (FIG. 9B) Quantification of phagocytosis of B cells treated with bestatin (20 μM), tosedostat (10 μM) or DMSO, or with an anti-CD24 antibody (clone SN3) or, as a control, IgG. Error bars indicate SD (n=3 technical replicates). (FIG. 9C) Quantification of phagocytosis of OVCAR8 cells treated with bestatin (20 μM), tosedostat (10 μM) or DMSO, or with an anti-CD24 antibody (clone SN3) or, as a control, IgG. Error bars indicate SD (n=3 technical replicates). P values were calculated using student t test or one-way ANOVA followed by Dunnett's multiple comparisons test. ***P<0.001, ****P<0.0001.

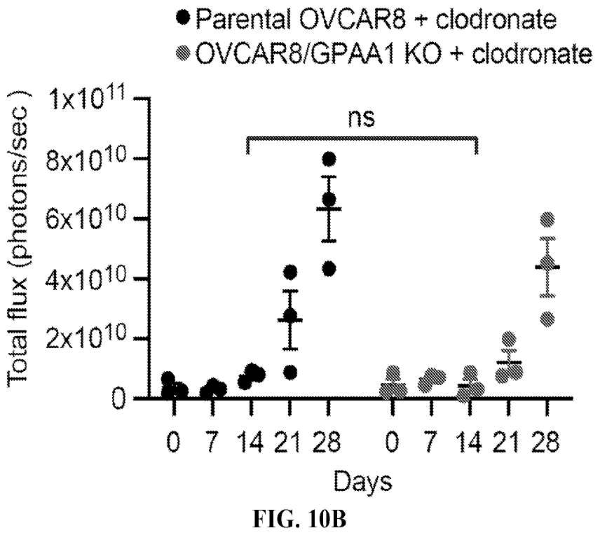

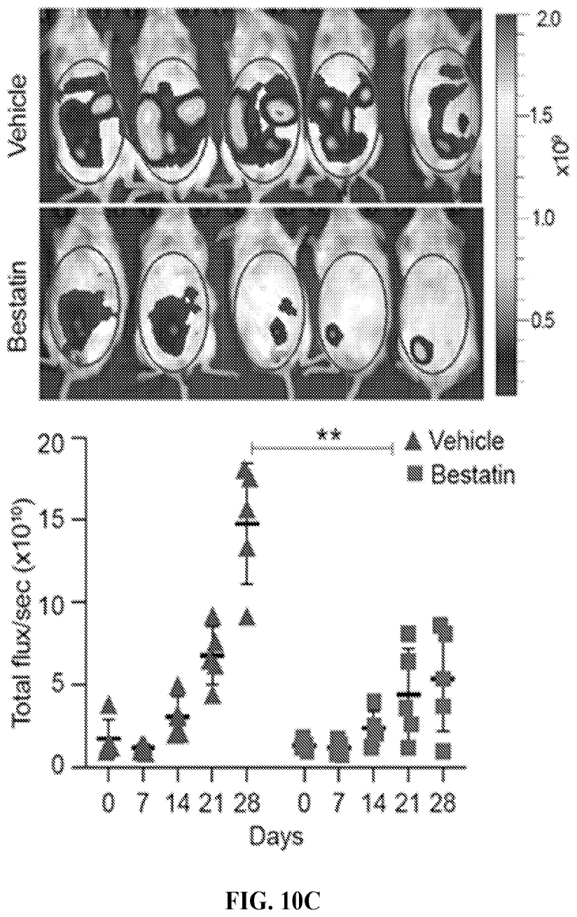

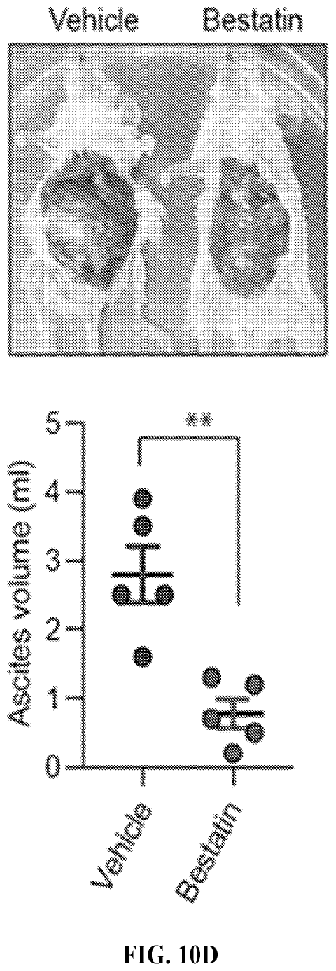

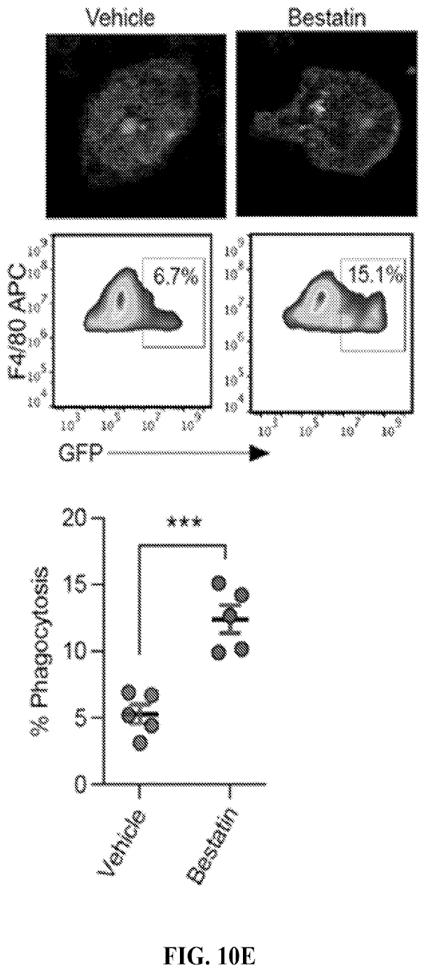

FIGS. 10A-10E: Genetic or pharmacological inhibition of GPAA1 suppresses growth of tumors derived from human ovarian cancer cells in xenograft mice. (FIG. 10A) Tumor growth, as monitored by in vivo bioluminescence imaging, in NSG mice intraperitoneally injected with parental or GPAA1 KO OVCAR8 cells. IVIS Spectrum images were captured 10 min after mice were intraperitoneally injected with firefly D-luciferin at a dose of 150 mg/kg. The total flux was calculated using Living Image 4.0 software. Error bars indicate SEM (n=3 mice per group). P value was calculated using two-way ANOVA. *P<0.02. (FIG. 10B) Tumor growth in NSG mice intraperitoneally injected with parental or GPAA1 KO OVCAR8 cells and treated with clodronate liposomes. Error bars indicate SEM (n=3 mice per group). P value was calculated using two-way ANOVA; the differences were not significant (ns). (FIG. 10C) Tumor growth in NSG mice treated with 100 mg/kg/d bestatin or vehicle (40% w/v 2-hydroxypropyl-beta-cyclodextrin). (Left), Representative images. (Right), Total flux. Error bars indicate SEM (n=5 mice per group). P value was calculated using two-way ANOVA. **P<0.01. (FIG. 10D) Ascites volume measurements from vehicle- or bestatin-treated mice following 10 days of treatment. (Top), Representative images of the peritoneal cavity of mice. (Bottom), Quantification. Error bars indicate SEM (n=5 mice per group). P value was calculated using an unpaired t-test. **P<0.01. (FIG. 10E) Phagocytosis assay on the aspirated ascites from vehicle- or bestatin-treated mice (n=5 per group). (Top), Representative flow cytometry plots. (Bottom), Quantification of F4/80+ GFP+ events. Error bars indicate SEM (n=5 mice per group). P value was calculated using an unpaired t-test. ***P<0.001.

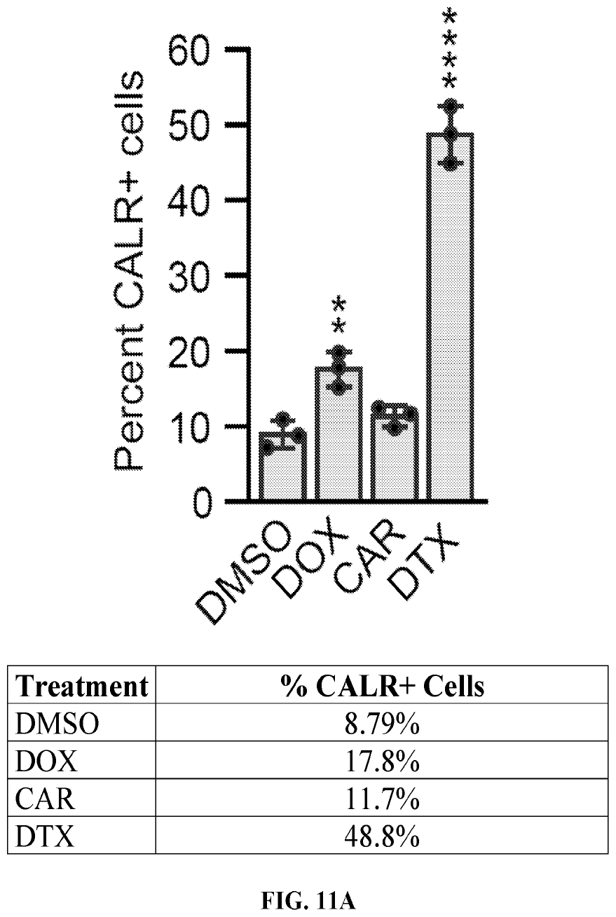

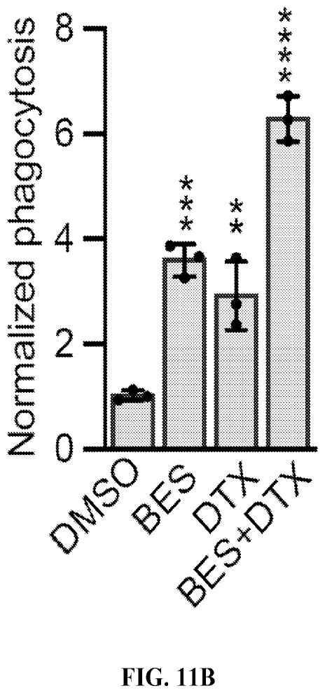

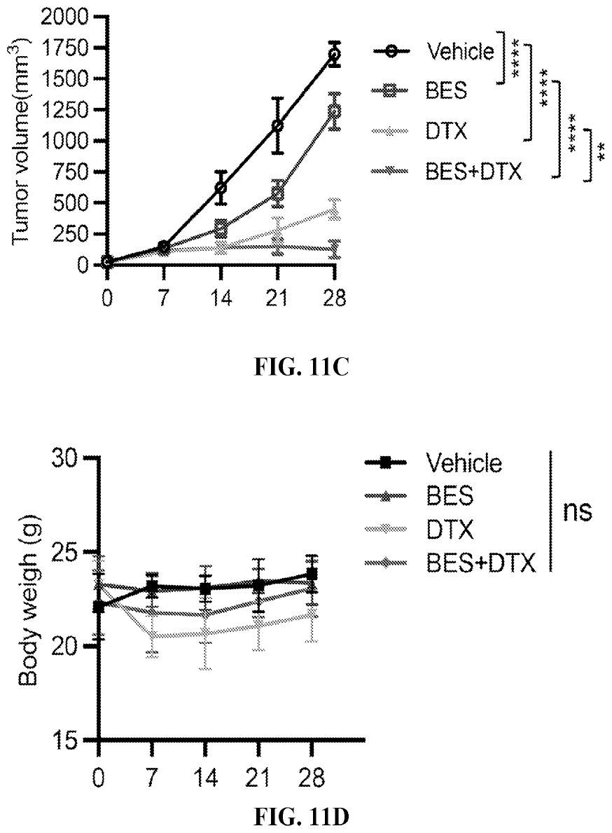

FIGS. 11A-11D: Docetaxel enhances the ability of bestatin promote macrophage-mediated phagocytosis of OVCAR8 cells. (FIG. 11A) Flow cytometry analysis of Calreticulin (CALR) in OVCAR8 cells treated with either doxorubicin (DOX, 50 nM), carboplatin (CAR, 100 nM) or docetaxel (DTX, 100 nM). DMSO was used as control. (FIG. 11B) Phagocytosis assay in OVCAR8 cells treated with bestatin or docetaxel or in combination. Error bars indicate SD (n=3 technical replicates). (FIG. 11C) Measurement of tumor growth. Error bars indicate SD (n=5 mice). (FIG. 11D) Measurement of animal weight. P values were calculated using two-way ANOVA followed by Turkey's multiple comparisons test. **P<0.01, ***P<0.001, ****P<0.0001.

FIG. 12: Suppression of ovarian tumor growth by bestatin and other aminopeptidase inhibitors. Bestatin (and certain other aminopeptidase inhibitors) bind to and inhibit GPAA1 thereby preventing attachment of a GPI lipid anchor to CD24, resulting in reduced CD24 cell surface expression. In the absence of the cell surface CD24 “don't eat me” signal, macrophages phagocytose and kill ovarian cancer cells, resulting in reduced tumor growth.

### DETAILED DESCRIPTION

The standard treatment for newly diagnosed ovarian cancer includes cytoreductive (debulking) surgery followed by chemotherapy, which consists of a combination of a platinum compound (usually cisplatin/Platinol or carboplatin/Paraplatin) and a taxane (such as paclitaxel/Taxol or docetaxel/Taxotere) (Armstrong et al., J Natl Compr Canc Netw 2021, 19(2):191-226). Despite a high response rate to first-line treatment, recurrence occurs in 25% of patients with early-stage disease and 70-80% of patients with advanced disease, with the majority developing platinum-resistant disease (Pokhriyal et al., Biomark Cancer 2019, 11:1179299X19860815). Thus, new, effective treatments for ovarian cancer are needed.

Over the past several years, new classes of targeted therapies and immunotherapies have been developed and approved to treat ovarian cancer. The anti-angiogenesis drug bevacizumab/Avastin, a monoclonal antibody that targets the VEGF/VEGFR pathway, is approved for patients with platinum-resistant recurrent ovarian cancer or newly-diagnosed advanced ovarian cancer in combination with chemotherapy (Haunschild and Tewari, Future Oncol 2020, 16(7):225-246). PARP inhibitors (such as olaparib/Lynparza) have recently been approved as a monotherapy or in combination with bevacizumab as a maintenance therapy in a subset of patients with BRCA-mutated or homologous recombination deficient (HRD)-positive advanced ovarian cancer following first-line treatment (Turcu et al., Oncologist 2021, 26(1):e164-e172). Generally, these treatments improve progression-free survival by only a few months with no significant effect on overall survival.

With regard to immunotherapies, epithelial ovarian cancers are considered potentially immunoreactive tumors due to the presence of tumor infiltrating lymphocytes, with the degree of infiltration correlating with improved survival (Zhang et al., NEngl J Med 2003, 348(3):203-13; Anadon et al., Cancer Cell 2022, 40(5):545-557.e13). However, to date, conventional immune checkpoint inhibitors targeting the PD-1/PD-L1 pathway have shown limited efficacy as single agents in clinical trials for ovarian cancer (Chardin and Leary, Front Oncol 2021, 11:795547), which is presumably due to low expression of PD-L1 in ovarian cancer (Barkal et al., Nature 2019, 572(7769):392-396). Moreover, although the anti-PD-1 monoclonal antibody pembrolizumab/Keytruda is an option for a subset of patients with advanced ovarian cancer with high microsatellite instability, DNA mismatch repair deficiency or high tumor mutational burden (Lemery et al., N Engl J Med 2017, 377(15):1409-1412), only about 10% of ovarian cancers fall into this category (Pal et al., Clin Cancer Res 2008, 14(21):6847-54).

Thus, there is a need for therapies targeting ovarian cancer. Accordingly, provided herein are methods for treating ovarian cancer that utilize an inhibitor of a GPI pathway protein.

### I. Inhibitors of GPI Pathway Proteins

About 150 different human proteins are anchored to the extracellular layer of a cell membrane via glycosylphosphatidylinositol (GPI). Such proteins are referred to as GPI-anchored proteins (GPI-APs). GPI-APs are involved in various cellular functions. Some GPI-APs such as CD24 can play a role in immune evasion by ovarian cancer cells. GPI-APs are synthesized in a complex process that is divided into three parts: biosynthesis, protein attachment to GPI, and GPI-AP remodeling. In some examples, GPI-APs synthesis involves at least 15 steps and more than 20 different proteins encoded by phosphatidyl inositol glycan (PIG) genes and post-GPI attachment to proteins (PGAP) genes.

As used herein, “GPI pathway protein” refers to any protein involved in the biosynthesis of GPI, attachment of GPI, and GPI-AP remodeling. Non-limiting examples of GPI pathway proteins that can be inhibited in methods described herein include glycosylphosphatidylinositol anchor attachment 1 (GPAA1), phosphatidylinositol glycan anchor biosynthesis class K (PIGK), phosphatidylinositol glycan anchor biosynthesis class P (PIGP), phosphatidylinositol glycan anchor biosynthesis class T (PIGT), and phosphatidylinositol glycan anchor biosynthesis class U protein (PIGU). Exemplary amino acid sequences of human GPAA1, PIGP, PIGT, and PIGU are provided in GenBank Accession Number NP_003792.1, NP_710148.1, NP_057021.2, and NP_536724.1, respectively. Methods described herein involve treating ovarian cancer using an inhibitor of GPAA1, PIGK, PIGP, PIGT, PIGU, or combinations thereof.

An inhibitor of any GPI pathway protein can be used in methods described herein. The term “inhibitor,” as used herein, refers to a molecule (e.g., a small molecule or a biological molecule) that blocks, inhibits, reduces (including significantly), or interferes with a GPI pathway protein (e.g., GPAA1, PIGK, PIGP, PIGT, PIGU) biological activity in vitro, in situ, and/or in vivo.

The term “inhibitor” implies no specific mechanism of biological action whatsoever, and expressly includes and encompasses all possible pharmacological, physiological, and biochemical interactions with a GPI pathway protein whether direct or indirect, and whether interacting with the GPI pathway protein or their substrates, or through another mechanism, and its consequences which can be achieved by a variety of different, and chemically divergent compositions.

In some examples, an inhibitor can be a molecule that inhibits or disrupts a GPI pathway protein itself (e.g., a human GPI pathway protein), a biological activity of a GPI pathway protein (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts GPAA1 itself (e.g., human GPAA1), a biological activity of GPAA1 (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts PIGK itself (e.g., human PIGK), a biological activity of PIGK (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts PIGP itself (e.g., human PIGP), a biological activity of PIGP (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts PIGT itself (e.g., human PIGT), a biological activity of PIGT (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts PIGU itself (e.g., human PIGU), a biological activity of PIGU (e.g., including but not limited to its ability to reduce surface expression of CD24), or the consequences of the biological activity to any meaningful degree, e.g., at least 10%, at least 20%, at least 30%, at least 40%, at least 50%, at least 60%, at least 70%, at least 80%, at least 90%, or more.

Non-limiting examples of an inhibitor of a GPI pathway protein for use in the methods described herein include a small molecule, an agent that inhibits expression of a GPI pathway protein (e.g., a nucleic acid molecule that inhibits the expression of the GPI pathway protein), an antibody targeting a GPI pathway protein, or a peptide that inhibits a GPI pathway protein (e.g., a peptide aptamer, a structural analog of the GPI pathway protein). In some embodiments, the inhibitor binds the GPI pathway protein (i.e., physically interacts with the GPI pathway protein), binds to a binding partner of the GPI pathway protein, and/or inhibits expression (i.e., transcription or translation) or processing of the GPI pathway protein.

Non-limiting examples of an inhibitor of GPAA1 for use in the methods described herein include a small molecule, an agent that inhibits expression of GPAA1 (e.g., a nucleic acid molecule that inhibits GPAA1 expression), anti-GPAA1 antibody, or a peptide that inhibits GPAA1 (e.g., a peptide aptamer, a GPAA1 structural analog). In some embodiments, the GPAA1 inhibitor binds GPAA1 (i.e., physically interacts with GPAA1), binds to a binding partner of GPAA1, and/or inhibits expression (i.e., transcription or translation) or processing of GPAA1.

Non-limiting examples of an inhibitor of PIGK for use in the methods described herein include a small molecule, an agent that inhibits expression of PIGK (e.g., a nucleic acid molecule that inhibits PIGK expression), anti-PIGK antibody, or a peptide that inhibits PIGK (e.g., a peptide aptamer, a PIGK structural analog). In some embodiments, the PIGK inhibitor binds PIGK (i.e., physically interacts with PIGK), binds to a binding partner of PIGK, and/or inhibits expression (i.e., transcription or translation) or processing of PIGK.

Non-limiting examples of an inhibitor of PIGP for use in the methods described herein include a small molecule, an agent that inhibits expression of PIGP (e.g., a nucleic acid molecule that inhibits PIGP expression), anti-PIGP antibody, or a peptide that inhibits PIGP (e.g., a peptide aptamer, a PIGP structural analog). In some embodiments, the PIGP inhibitor binds PIGP (i.e., physically interacts with PIGP), binds to a binding partner of PIGP, and/or inhibits expression (i.e., transcription or translation) or processing of PIGP.

Non-limiting examples of an inhibitor of PIGT for use in the methods described herein include a small molecule, an agent that inhibits expression of PIGT (e.g., a nucleic acid molecule that inhibits PIGT expression), anti-PIGT antibody, or a peptide that inhibits PIGT (e.g., a peptide aptamer, a PIGT structural analog). In some embodiments, the PIGT inhibitor binds PIGT (i.e., physically interacts with PIGT), binds to a binding partner of PIGT, and/or inhibits expression (i.e., transcription or translation) or processing of PIGT.

Non-limiting examples of an inhibitor of PIGU for use in the methods described herein include a small molecule, an agent that inhibits expression of PIGU (e.g., a nucleic acid molecule that inhibits PIGU expression), anti-PIGU antibody, or a peptide that inhibits PIGU (e.g., a peptide aptamer, a PIGU structural analog). In some embodiments, the PIGU inhibitor binds PIGU (i.e., physically interacts with PIGU), binds to a binding partner of PIGU, and/or inhibits expression (i.e., transcription or translation) or processing of PIGU.

Any small molecule suitable for inhibiting a GPI pathway protein (e.g., GPAA1, PIGK, PIGP, PIGT, PIGU) can be used in methods described herein. The term “small molecule inhibitor of a GPI pathway protein” refers to small organic compounds, inorganic compounds, or any combination thereof that inhibits or reduces a biological activity of a GPI pathway protein. In some embodiments, a small molecule inhibitor of a GPI pathway protein used in the methods described herein inhibits a biological activity of a GPI pathway protein by at least 10% (e.g., at least 20%, at least 30%, at least 40%, at least 50%, at least 60%, at least 70%, at least 80%, at least 90%, or more). Small molecule inhibitors can be used in methods described herein in the free form, as a salt thereof, a prodrug derivative thereof, or combinations thereof.

In some embodiments, the small molecule inhibitor of a GPI pathway protein is a metallo-aminopeptidase inhibitor. Non-limiting examples of a metallo-aminopeptidase inhibitor for use in methods described herein includes bestatin, LYP, LYP3, tosedostat, ARM1, or combinations thereof.

Any agent suitable for inhibiting expression of a GPI pathway protein (e.g., GPAA1, PIGK, PIGP, PIGT, PIGU) can be used in methods described herein. In some embodiments, an agent that inhibits expression of a GPI pathway protein (e.g., GPAA1, PIGP, PIGT, PIGU) used in the methods described herein inhibits expression of the GPI pathway protein by at least 10% (e.g., at least 20%, at least 30%, at least 40%, at least 50%, at least 60%, at least 70%, at least 80%, at least 90%, or more). In some examples, agents that inhibit expression of a GPI pathway protein are nucleic acid molecules such as short interfering nucleic acid (siNA), short interfering RNA (siRNA), double-stranded RNA (dsRNA), micro-RNA (miRNA), antisense oligonucleotides (ASOs), targeted protein degraders (PROTACs), and short hairpin RNA (shRNA) molecules. Such nucleic acid molecules can include non-naturally-occurring nucleobases (e.g., modified nucleobases), sugars (e.g., substituted sugar moieties), and/or covalent internucleoside linkages (e.g., modified backbones). Exemplary nucleic acid sequences of human GPAA1, PIGK, PIGP, PIGT, and PIGU are provided in GenBank Accession Numbers NM_003801.4, NM_005482.2, NM_153681.2, NM_015937.6, and NM_080476.5, respectively.

In some examples, the inhibitor of a GPI pathway protein can be one or more molecules that disrupt the gene encoding the GPI pathway protein (e.g., the GPAA1 gene, the PIGK gene, the PIGP gene, the PIGT gene, the PIGU gene). Any molecule(s) known in the art can be used to disrupt the gene encoding the GPI pathway protein including gene editing molecules such as guide RNA (gRNA) and clustered regularly interspaced short palindromic repeat (CRISPR)-associated 9 (Cas9) nuclease.

In some examples, the inhibitor of a GPI pathway protein to be used in methods described herein can be an antibody targeting the GPI pathway protein (e.g., an anti-GPAA1 antibody, an anti-PIGK antibody, an anti-PIGP antibody, an anti-PIGT antibody, an anti-PIGU antibody). An antibody targeting a GPI pathway protein is an antibody capable of binding to the GPI pathway protein, which can inhibit a biological activity of the GPI pathway protein and/or downstream pathway(s) mediated by signaling of the GPI pathway protein. In some embodiments, an antibody targeting a GPI pathway protein used in the methods described herein inhibits a biological activity of the GPI pathway protein and/or downstream pathway(s) mediated by signaling of the GPI pathway protein by at least 10% (e.g., at least 20%, at least 30%, at least 40%, at least 50%, at least 60%, at least 70%, at least 80%, at least 90%, or more).

An antibody is an immunoglobulin molecule capable of specific binding to a target, such as carbohydrate, polynucleotide, lipid, polynucleotide, lipid, polypeptide, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof such as Fab, Fab′, F(ab′)₂, Fv, single chain (scFv), mutants thereof, fusion proteins comprising antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody targeting a GPI pathway protein can be an antibody of any class, such as IgD, IgE, IgG, IgA, or IgM (or sub-class thereof), or the antibody targeting the GPI pathway protein need not be of any particular class.

In some examples, the inhibitor of a GPI pathway protein to be used in methods described herein can be a peptide inhibitor. In some embodiments, a peptide inhibitor used in the methods described herein inhibits a biological activity of a GPI pathway protein and/or downstream pathway(s) mediated by signaling of a GPI pathway protein by at least 10% (e.g., at least 20%, at least 30%, at least 40%, at least 50%, at least 60%, at least 70%, at least 80%, at least 90%, or more).

Any of the polynucleotides and polypeptides described herein for inhibiting a GPI pathway protein can be included in a delivery vehicle. The delivery vehicle can be of viral (e.g., viral vectors) or non-viral origin (e.g., eukaryotic cell delivery vehicles). Viral vectors are known in the art and include, but are not limited to, retroviral vectors, lentiviral vectors, adenoviral vectors, adeno-associated viral vectors, herpes simplex viral vectors, and vaccinia viral vectors. Expression of coding sequences in the delivery vehicle can be induced using endogenous or heterologous promoters and/or enhances. Expression of the coding sequence can be either constitutive or regulated.

Any inhibitor (e.g., those described herein) can be mixed with a pharmaceutically acceptable excipient (carrier) to form a pharmaceutical composition for use in treating an ovarian cancer. “Acceptable” means that the excipient must be compatible with the inhibitor (and preferably, capable of stabilizing the inhibitor) and not deleterious to the subject to be treated. Pharmaceutically acceptable excipients (carriers), including buffers, are well known in the art. See, e.g., Remington: The Science and Practice of Pharmacy 20^{th} Ed. (2000) Lippincott Williams and Wilkins, Ed. K. E. Hoover.

### II. Use of Inhibitors of GPI Pathway Proteins for Treating Ovarian Cancer

Aspects of the present disclosure provide methods for treating ovarian cancer using an inhibitor of a GPI pathway protein. Non-limiting examples of ovarian cancer include epithelial ovarian cancer, germ cell ovarian cancer, stromal cell ovarian cancer, small cell carcinoma of the ovary (SCCO), and combinations thereof.

Epithelial ovarian cancer arises from the epithelium of the ovary and it is the most common ovarian cancer. Non-limiting examples of epithelial ovarian cancer include low-grade serous ovarian carcinoma (LGSOC), clear cell carcinoma (CCC), endometrioid carcinoma, mucinous carcinoma, squamous cell carcinoma (SCC), borderline epithelial ovarian tumors, borderline endometrioid ovarian tumors, and combinations thereof.

To practice the methods disclosed herein, an effective amount of a composition comprising an inhibitor (e.g., a pharmaceutical composition comprising an inhibitor) can be administered to a subject (e.g., a human patient) having or at risk for having ovarian cancer via a suitable route (e.g., oral administration).

The term “subject” refers to a subject who needs treatment as described herein. In some embodiments, the subject is a human (e.g., a human patient) or a non-human mammal (e.g., cat, dog, horse, cow, goat, or sheep). A human subject who needs treatment can be a human patient having, suspected of having, or at risk for having ovarian cancer.

In some embodiments, a subject to be treated by methods described herein can be a subject having a chemo-resistant ovarian cancer. As used herein, a chemo-resistant ovarian cancer refers to an ovarian cancer that returns following treatment with a chemotherapy.

In some embodiments, the subject to be treated by the methods described herein can be a human subject having tumor cells that express one or more of CD24, GPAA1, PEPT1, and PEPT2. Tumor cells can be identified as expressing one or more of CD24, GPAA1, PEPT1, and PEPT2 using any method known in the art, e.g., an immune assay such as immunohistochemistry (IHC) or flow cytometry.

Any of the methods described herein can further comprise a step of identifying a subject for treatment based on presence and/or level of tumor cells expressing one or more of CD24, GPAA1, PEPT1, and PEPT2 in the subject. In some embodiments, presence and/or level of tumor cells expressing one or more of CD24, GPAA1, PEPT1, and PEPT2 is determined in a biological sample (e.g., a tissue sample) obtained from the subject.

In some embodiments, at least 5% (e.g., at least 10%, at least 15%, at least 20%) of tumor cells in the biological sample obtained from the human subject express CD24. In some embodiments, at least 5% (e.g., at least 10%, at least 15%, at least 20%) of tumor cells in the biological sample obtained from the human subject express GPAA1. In some embodiments, at least 5% (e.g., at least 10%, at least 15%, at least 20%) of tumor cells in the biological sample obtained from the human subject express PEPT1 and/or PEPT2.

A subject having ovarian cancer can be identified by routine medical examination, e.g., medical examination (e.g., pelvic exam), laboratory tests (e.g., blood tests), or imaging tests (e.g., ultrasound, CT scans). Such a subject can exhibit one or more symptoms associated with ovarian cancer, e.g., abdominal bloating or swelling, weight loss, discomfort in the pelvic area, fatigue, back pain, constipation, or combinations thereof. Alternatively, or in addition to, such a subject can have one or more risk factors for ovarian cancer, e.g., family history, being overweight or obese, talking hormone therapy after menopause, having children later or never having a full-term pregnancy, age, or combinations thereof.

An effective amount refers to the amount of an inhibitor of a GPI pathway protein needed to prevent or alleviate at least one or more signs or symptoms of an ovarian cancer, and relates to a sufficient amount of an inhibitor of a GPI pathway protein that provides the desired effect, e.g., to treat a human subject having an ovarian cancer. An effective amount also includes an amount sufficient to prevent or delay the development of a symptom of an ovarian cancer, alter the course of a symptom of an ovarian cancer (e.g., slow the progression of a symptom of an ovarian cancer), or reverse a symptom of an ovarian cancer.

In some embodiments, dosages of an inhibitor as described herein can be determined empirically in individuals who have been given one or more administration(s) of the inhibitor. For example, individuals are given incremental dosages of the inhibitor, and an indicator and/or a symptom of an ovarian cancer can be followed to assess efficacy of the inhibitor.

In some embodiments, the appropriate dosage of an inhibitor will depend on the specific inhibitor(s) (or pharmaceutical compositions thereof) used, the type and severity of ovarian cancer, previous therapy, the patient's clinical history and response to the inhibitor(s), and the discretion of the healthcare practitioner.

As used herein, the term “treating” refers to the application or administration of a composition including one or more inhibitors of a GPI pathway protein to a subject who has an ovarian cancer, a symptom of an ovarian cancer, and/or a predisposition toward an ovarian cancer, with the purpose to cure, heal, alleviate, relieve, alter, remedy, ameliorate, improve, or affect the ovarian cancer, the symptom of the ovarian cancer, and/or the predisposition toward the ovarian cancer.

Alleviating an ovarian cancer includes delaying the development or progression of the disease, and/or reducing disease severity. Alleviating the disease does not necessarily require curative results.

As used herein, “delaying” the development of an ovarian cancer means to defer, hinder, slow, retard, stabilize, and/or postpone progression of the ovarian cancer. This delay can be of varying lengths of time, depending on the history of the ovarian cancer and/or individuals being treated. A method that “delays” or alleviates the development of an ovarian cancer and/or delays the onset of the ovarian cancer is a method that reduces probability of developing one or more symptoms of the ovarian cancer in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the ovarian cancer. Development of the ovarian cancer can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence” of an ovarian cancer includes initial onset and/or recurrence.

In some embodiments, the inhibitor of the GPI pathway protein is administered to a subject in an amount sufficient to reduce cell surface expression of CD24 by at least 10% (e.g., at least 20%, at least 30%, at least 40%, at least 50%, at least 60%, at least 70%, at least 80%, at least 90%, or more).

In some embodiments, the inhibitor of the GPI pathway protein is administered to a subject in an amount sufficient to increase phagocytosis of ovarian cancer cells by at least 10% (e.g., at least 20%, at least 30%, at least 40%, at least 50%, at least 60%, at least 70%, at least 80%, at least 90%, or more).

In some embodiments, the inhibitor of the GPI pathway protein is administered to a subject in an amount sufficient to reduce tumor growth by at least 10% (e.g., at least 20%, at least 30%, at least 40%, at least 50%, at least 60%, at least 70%, at least 80%, at least 90%, or more).

The inhibitor of the GPI pathway protein can be administered using any suitable method for achieving delivery of the inhibitor to the subject in need thereof. In some embodiments, the inhibitor can be administered orally, topically, nasally, parenterally, buccally, or by inhalation. Parenteral administration includes, but is not limited to, subcutaneous, intracutaneous, intravenous, intramuscular, or intrasynovial injection or infusion techniques.

The particular dosage regimen, e.g., dose, timing, and repetition, used in methods described herein will depend on the particular subject and that subject's medical history.

In some embodiments, more than one inhibitor of a GPI pathway protein can be administered to a subject in need thereof (e.g., a small molecule inhibitor and a peptide inhibitor are administered to the subject). The inhibitor of the GPI pathway protein can be the same type or different from each other. At least one, at least two, at least three, at least four, or at least five different inhibitors of GPI pathways proteins can be co-administered. In such instances, inhibitors can have complementary activities that do not adversely affect each other. Inhibitors can also be used in conjunction with other agents that serve to enhance and/or complement the effectiveness of the inhibitor.

In some embodiments, the inhibitor of the GPI pathway protein is administered one or more times to the subject. Alternatively, or in addition, the inhibitor can be administered as part of a combination therapy comprising an inhibitor a GPI pathway protein and an additional anti-cancer therapy such as a chemotherapy, a surgical therapy, or both. The term combination therapy, as used herein, embraces administration of the inhibitor and the additional anti-cancer therapy in a sequential manner, that is wherein each therapy is administered at a different time, as well as administration of these therapies, or at least two of the therapies, in a substantially simultaneous manner.

The following examples are provided to better illustrate the claimed invention and are not to be interpreted as limiting the scope of the invention in any way. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

Single-Cell Analysis of Innate Immune Components in Ovarian Cancer: Published single-cell RNA-seq data from ascites specimens collected from 11 HGSOC patients i5 (GSE146026; Izar et al., Nat Med 2020, (8):1271-1279) was analyzed using BBrowser, a free software program for exploring single-cell omics datasets that is available from BioTuring Inc. (bioturing.com/bbrowser). UMAP plots were generated using default parameters.

Cell Lines and Culture Conditions: Human cell lines were obtained from the following sources, OVCAR8 (Creative Biolabs, Cat #IOC-ZP305), SKOV3 (ATCC, HTB-77), OVCAR4 (Sigma-Aldrich, Cat #SCC258), NCI-ADR-RES(RRID:CVCL_1452), IGROV1 (Sigma-Aldrich Cat #SCC203) MCF7 (ATCC, HTB-22) and HEK-293T (ATCC, CRL-3216) Primary B cells (refer to the isolation of B cells from human PBMCs described below). OVCAR8, SKOV3, IGROV1, OVCAR4 and NCI-ADR-RES cells were cultured in RPMI 1640 medium (Gibco, Cat #11875093). MCF7 and HEK293T cell lines were maintained in Dulbecco's Modified Eagle Medium (DMEM) with high glucose (HyClone™, Cat #SH30022.01). Media were supplemented with 10% (v/v) fetal bovine serum (FBS) (Atlanta Biologicals, Cat #S11550), sodium pyruvate (Thermo Fisher Scientific, Cat #11360070) non-essential amino acids (Gibco, Cat #11140050), 100 units/ml penicillin and 100 g/ml streptomycin (Gibco, Cat #15140122). All cell lines were cultured at 37° C. and 5% CO₂. Sublines derived from OVCAR8 were also cultured in the same conditions as the parental cell line.

Lentivirus Packaging, Transduction, and shRNA Knockdown: For packaging lentiviral shRNAs, 1.0×10⁶ HEK293T cells were seeded in 6-well plates and incubated overnight until cells reached ~70-80% confluency. Fresh OptiMEM medium (Gibco, Cat #31985070) was added 1 hour before transfection. Lentiviral transgene vectors (1 μg, see below) and packaging plasmids psPAX2 (a gift a from Didier Trono [Addgene plasmid #12260]; 1 g) and pMD2.G (a gift from Didier Trono [Addgene plasmid #12259]; 0.5 μg) were mixed in a 2:2:1 ratio in 100 μl EC buffer (QIAGEN) in a sterile eppendorf tube. Transfection was performed using Effectene transfection reagent (QIAGEN, cat #301425). Next day the medium was replaced to remove DNA complexes. 48 h post-transfection medium containing lentiviral particles was collected and filtered through a 0.45 μm filter. The viral supernatant was either used immediately or aliquoted and stored at −80° C. until further use. For shRNA knockdown, 1×10⁵ cells per well were seeded in 6-well plates and transduced with 500 μl lentivirus particles (~2×10⁶ TU/ml) packaged with an shRNA-expressing TRC lentivirus vector (listed in Table 1 and obtained from the UMass Chan Medical School RNAi Core Facility or Sigma) in a total volume of 1 ml of appropriate medium supplemented with 8 μg/ml polybrene (Sigma, Cat #1003). Medium was replaced after overnight incubation to remove polybrene and viral particles, and cells were incubated for another 24 hours and then subjected to puromycin (Invivogen, Cat #ant-pr-5b) selection (2 μg/ml) for 3-5 days.

Cell Viability Assay: OVCAR8 cells were transduced with a lentivirus expressing non-silencing (NS) or CD24 shRNA (Table 1) and analyzed for cell viability using PrestoBlue Cell Viability Reagent (Invitrogen™, Cat #A13261) as per the manufacturer's instructions, and relative cell growth was determined. Relative cell viability of OVCAR8 or SKOV3 cells treated with either DMSO or bestatin (Ubenimex) (SelleckChem, Cat #S1591) at 5, 10 and 20 mM was also determined using a PrestoBlue assay.

For crystal violet staining 1×10⁴ cells were seeded in 6 well plate and cultured for 5 days. Cells were washed with PBS and fixed using 1 ml of 4% paraformaldehyde for 20 minutes at room temperature. Following two rinses with PBS, the cells were stained with 0.5 ml of 0.1% crystal violet solution (in 10% ethanol) for 20 minutes. The cells were rinsed twice with PBS, and the plate was scanned.

CRISPR/Cas9 Screen: To generate a stable Cas9-expressing OVCAR8 cell line, the plasmid lentiCas9-Blast (a gift from Fen Zhang [Addgene plasmid #52962]; Sanjana et al., Nat Methods 2014, 11(8):783-784) was packaged into a lentivirus as described above, and the viral supernatant was used to transduce OVCAR8 cells. Cells were selected for 5 days with blasticidin (5 μg/ml) and single-cell clones were isolated and tested for Cas9 gene editing efficiency using the Cas9 reporter vector pXPR_011 (a gift from John Doench and David Root [Addgene plasmid #59702]; Doench et al., Nat Biotechnol 2014, 32(12):1262-7). For the screen, 80×10⁶ OVCAR8/Cas9 cells were transduced with the Human CRISPR Knockout Pooled Library (Brunello) (a gift from David Root and John Doench [Addgene #73178]; Doench et al., Nat Biotechnol 2016, 34(2):184-191) at a multiplicity of infection (MOI) of 0.5. Cells were selected with 2 μg/ml puromycin for 15 days, and then stained with an APC-conjugated anti-human CD24 antibody (BioLegend, Cat #311118, clone ML5) for 30 min on ice in the dark. Five to 10 min prior to FACS, 7-AAD Viability Staining Solution (BioLegend, Cat #420404) was used to exclude dead cells from the analysis. At least 100×10⁶ CRISPR-edited cells were FACS sorted to isolate the CD24^{low} (defined as cells with the 5% lowest CD24 staining) and 7AAD^{neg} population. Total genomic DNA was extracted from the CD24^{low} and unsorted populations and 20 μg used to prepare a next-generation sequencing (NGS) library as previously described (Evers et al., Nat Biotechnol 2016, 34(6):631-3). Illumina NGS was performed, and Bowtiel was used to map the reads to the sgRNA sequences in the human Brunello library with the default parameter settings except-m 1-best-v 2 (Langmead et al., Genome Biol 2009, 10(3):R25). sgRNAs with a BH-adjusted p-value <0.05 (Benjamini and Hochberg J Roy Statist Ser B 1995) and odds ratio ≥2 using Fisher Exact test were considered as significantly enriched.

Gene Ontology Analysis: Gene Ontology (GO) enrichment analysis was performed using Bioconductor package ChIPpeakAnno (Zhu et al., BMC Bioinformatics 2010, 11:237; Zhu, Methods Mol Biol 2013, 1067:105-24). GO terms with p-value <0.001 were considered significant.

CRISPR Knockout Generation: For GPAA1 and CD24 CRISPR-mediated knockouts, sgRNAs targeting the GPAA1 and CD24 genes (Table 2) were cloned into the vector lenti-CRISPRv2 (a gift from Feng Zhang [Addgene #52961]; Sanjana et al., Nat Methods 2014, 11(8):783-784), which contains hSpCas9. The vector was digested with BsmBI, and a pair of annealed oligos was cloned into the sgRNA scaffold. The oligos were designed based on the target site sequence (20 bp) and flanked on the 3′ end by a 3 bp NGG PAM sequence. The lenti-CRISPRv2-GPAA1-sgRNA and lenti-CRISPRv2-CD24-sgRNA constructs were amplified by transformation in recombination deficient, chemically competent E. coli (Stbl3) bacteria (propagated from Invitrogen Cat #C737303). The plasmid DNA was isolated using a Thermo fisher PureLink HiPure Plasmid Midiprep Kit (Cat #K210004). Lenti-CRISPRv2-GPAA1-sgRNA virus was packed as described above. Cells were transduced with lentiviral particles and selected in puromycin (2 mg/ml) for 10 days. Single cell clones were isolated using serial dilution method in 96-well plates. Individual single cell clones were grown and knockout was confirmed by immunoblotting using a polyclonal GPAA1 or CD24 antibody.

Flow Cytometry Analysis: For cell surface protein staining, 1×10⁵-1×10⁶ cells were first incubated with Fc receptor blocking solution, Human TruStain FcX™ (BioLegend, Cat #422301) for 10 minutes at room temperature. Cells were washed once in FACS buffer (1×PBS, 1.0% bovine serum albumin (BSA) (Sigma) and 0.2% sodium azide (Sigma), and then incubated with antibodies (given below) for 30 minutes at 4° C. in the dark. Cells were also stained with 7-AAD (Biolegend, Cat #420404) for dead cell exclusion, and flow cytometry analysis was performed using a Bio-Rad ZE5 Cell Analyzer. Following antibodies used in this study (all antibodies were used at 1:50 dilution unless otherwise noted): APC anti-human CD24 (BioLegend, Cat #311118, clone ML5), PE anti-human CD24 (eBioscience, Cat #12-0247-42, clone SN3), APC anti-human CD47 (BioLegend, Cat #323124, clone CC2C6), APC anti-human § 2-microglobulin (BioLegend, Cat #395711, clone A17082A), PE anti-human HLA-A2 (MHC-class I) (BioLegend, Cat #343305, clone BB7.2), APC anti-human CD11b (BioLegend, Cat #982604, clone ICRF44), APC anti-mouse F4/80 antibody (BioLegend, Cat #123116, clone BM8), APC-streptavidin (Thermo Fisher Scientific, Cat #SA1005, 1:100 dilution), PE anti-Calreticulin (Invitrogen, Catalog #MA5-15382, 1:200 dilution). For intracellular staining, cells were fixed with 1% paraformaldehyde for 5 min and permeabilized using the True-Nuclear Transcription Factor Buffer Set (BioLegend, Cat #424401) before the addition of the antibody.

Quantitative RT-PCR: Total RNA was extracted from cells using Trizol (Invitrogen, Cat #15596). cDNA was synthesized using Proto Script II reverse transcription kit (NEB, Cat #E6560) and real-time PCR reactions were performed using Quant Studio 3 (Applied Biosystems by Thermo Scientific) using primer sequences listed in Table 3. Expression was normalized to that of GAPDH. Experiments were performed three independent times and the results from one representative experiment are shown in technical triplicate.

Immunoblot Analysis: Cells seeded in 6-well plates were harvested and lysed in RIPA buffer (1% Triton-X100, 0.1% SDS, 0.5% deoxycholic acid, 20 mM Tris, pH 7.5, 10% Glycerol) containing 1× protease inhibitor cocktail (Roche, Cat #11 873 580 001) and 1 mM PMSF. Total lysates (30 μg) were run on 10% SDS-PAGE and transferred to nitrocellulose membrane. Membranes were incubated with anti-GPAA1 (1:1000 dilution; Invitrogen, Cat #PA5-100548), anti-CD24 (1:1,000 dilution; Abcam, Cat #ab290730), anti-PEPT1 (1:1,000 dilution; Santacruz, Cat #sc-37374271), anti-PEPT2 (1:1,000 dilution; Novus Biologicals, Cat #NBP1-59626) and anti-b-actin (1:2000 dilution; Sigma, Cat #A2228) antibodies overnight at 4° C. The blots were imaged by exposing to x-ray film. Experiments were conducted three independent times and the results from one representative experiment are shown.

Analysis of GPAA1 Genomic Amplification: Pan-cancer analysis of genomic amplification of GPAA1 was performed using the online cBioPortal database (www.cbioportal.org).

Kaplan Meier Analysis: To generate the survival curves for patients expressing high (upper 25%) versus low (other 75%) levels of GPAA1 or CD24, the interactive web server Online consensus Survival analysis for Ovarian cancer (OSov) (Yan et al., Biology (Basel) 2021, 11(1):23) found at the Long-term Outcome and Gene Expression Profiling Database of pan-cancers (LOGpc) (bioinfo.henu.edu.cn/OV/OVList.jsp) was used to query the datasets GSE63885 (for GPAA1) and GSE8841 (for CD24) (Lisowska et al., Front Oncol 2014, 4:6).

Confocal Microscopy: To prepare the cells for confocal microscopy, cells were grown on coverslips and fixed with 4% paraformaldehyde (PFA) (Sigma Aldrich Cat #158127) for 20 minutes at room temperature followed by three washes with cold 1λPBS. The cells were then permeabilized with 0.3% Triton X-100 (Sigma Aldrich, Cat #9036-19-5) for 10 minutes, washed three times with cold PBS, blocked for 1 hour at room temperature with 10% goat serum (Gibco Cat #16210064) in PBS, and then incubated at 4° C. overnight with recombinant anti-CD24 primary antibody (Abcam Cat #ab202073, clone EPR19925). The cells were then washed three times with PBS, incubated with a fluorescently conjugated secondary antibody (goat anti-rabbit IgG H&L (Alexa Fluor 594) (Abcam Cat #ab150080) for 1 hour at room temperature, and then washed again three times with PBS. For ER staining, cells were incubated with ER-Tracker Blue-White DPX (Thermo Fisher Scientific, Cat #E12353) in PBS for 10 minutes at room temperature. Thereafter, the cells were washed three times with PBS before being mounted in ProLong glass antifade mounting medium (Thermo Fisher Scientific, Cat #P36980). Images were taken using a Leica SP8 Laser Scanning Confocal Fluorescence microscope. Images were acquired through a 100× objective lens (oil). The z-stack images were taken with a 0.2 mm thick frame. Images were processed and analyzed using Fiji software.

Small Molecule Inhibitor and Drug Treatments: OVCAR8 or SKOV3 cells were treated for 3 days with bestatin (Ubenimex) (SelleckChem, Cat #S1591) at 5, 10 or 20 μM (as indicated in the figure legends), Tosedostat (Medchem Express, Cat #HY-14807) at 5 μM or 10 μM (as indicated in the figure legends) or ARM-1 (Medchem Express, Cat #HY-W027340) at 1, or 2 μM (as indicated in the figure legends). Inhibitors were replenished every 24 hours. DMSO (Sigma Aldrich, Cat #D8418) was used as a vehicle. OVCAR8 cells were treated with Docetaxel (SelleckChem, Cat #S1148) at 100 nM or Carboplatin (SelleckChem, Cat #S1215 at 100 nM or Doxorubicin (SelleckChem, Cat #E2516) at 50 nM for 12 hours.

CETSA: CETSA assays were performed as described previously (Jafari et al., Nat Protoc 2014, 9(9):2100-22). In brief, lox 10⁶ OVCAR8 cells were treated with DMSO or 100 μM bestatin for 2 hours. Cells (50 μl) were aliquoted in PCR tubes and incubated at room temperature (25° C.) or at a range of temperatures (37° C. to 65.2° C.) in a thermal cycler for 3 min and cooled to 4° C. Cells were lysed using Alpha SureFire Ultra Lysis Buffer (PerkinElmer, Cat #ASLU-LB) supplemented with Halt Protease Inhibitor Cocktail (ThermoFisher Scientific, Cat #78430). Insoluble proteins were separated by centrifugation at 15000×g for 30 mins at 4° C. Equal volumes of each sample were separated by SDS-PAGE and detected by immunoblotting blotting using an anti-GPAA1 polyclonal antibody (Invitrogen, Cat #PA5-100548). Three independent experiments were performed. Densitometry of the blots was performed using ImageJ software and the graph was plotted using GraphPad Prism 9.0.

Macrophage Generation and Stimulation: Primary human donor-derived macrophages were generated as described previously (Martinez, Curr Protocol Immunol 2012, Chapter 14:14.28.1-14.28.23). In brief, Leukopaks from anonymous donors were obtained from the Rhode Island Blood (Providence, RI) and peripheral blood monocytes (PBMCs) were isolated by density gradients centrifugation using Ficoll Paque Plus (Sigma-Aldrich, Cat #17-1440-02) followed by RBC lysis using RBC lysis buffer (eBioscience, Cat #00-4333-57). For monocyte isolation, PBMCs were incubated in Monocyte Attachment Medium (Sigma-Aldrich, Cat #C-28051) for 1 hour at 37° C. and 5% CO₂ and then washed three times in Iscove's Modified Dulbecco's Medium (IMDM) (Gibco, Cat #12440053) to remove non-adherent cells. The remaining monocytes were detached and seeded in 96-well plates and cultured at 37° C. and 5% CO₂ in IMDM supplemented with 10% FBS (Atlanta Biologicals, Cat #S11550) and 20 ng/ml recombinant human M-CSF (Peprotech, Cat #AF-300-25). After 4 days monocytes were then treated with 20 ng/ml recombinant human IL-4 (Peprotech, Cat #200-04) and 10 ng/mL recombinant human IL-10 (PeproTech, Cat #200-10) for another 3-4 days to obtain M2 polarized macrophages. Macrophage differentiation was confirmed morphologically using microscopy. M2-macrophage polarization was confirmed by immunophenotyping of the M2 marker CD206 using PE-conjugated anti-human anti-CD206 antibody (BioLegend, Cat #321106, clone 15-2, 1:100 dilution).

Primary B-cell Isolation and Culture: Pan B cells were isolated by negative selection from PBMCs isolated from Leukopaks (as described above) using a MojoSort Human Pan B Cell Isolation Kit (BioLegend, Cat #480081) according to the manufacturer's instructions. The purity of B cells was confirmed to be >95% by flow cytometry using a FITC anti-human CD19 antibody (BioLegend, Cat #302205, clone HIB19). B cells were cultured in RPMI 1640 medium supplemented with 5% human serum (Sigma-Aldrich, Cat #H6914), MEM non-essential amino acids solution (Gibco, Cat #1140050), and recombinant human IL-2 (50 ng/ml) (PeproTech, Cat #200-02) and IL-4 (10 ng/ml) (PeproTech, Cat #200-04).

In vitro Phagocytosis Assays: To facilitate the quantification of phagocytic events, we adapted a microscopy-based fluorescence phagocytosis assay (Miksa et al., J Immunol Methods 2009, 342(1-2):71-7; Lindner et al., Biotechniques 2020, 68(5):245-250) for use with a Celigo imaging cytometer. In brief, OVCAR8 cells were stably transduced with a lentivirus expressing Emerald-Green-luciferase (pLenti-Emerald-Green-luc, a gift from Jonathan M Hernandez, NCI) OVCAR8/EmGFP-luc cells or B cells (isolated as described above) were pre-treated with either DMSO, bestatin (10 or 20 μM) for 2-3 days, ARM1 (2 μM) or tosedostat (10 μM) for 24 hour, Docetaxel (100 nM) or Carboplatin (100 nM) or Doxorubicin (50 nM) for 12 hours or IgG (Novus Biologicals, Cat #MAB11012-100, clone 1268C) or anti-CD24 antibody (Novus Biologicals, Cat #NB100-64861, clone SN3) for 2 hours. Cells were incubated with pHrodo Red, SE (Invitrogen, Cat #P36600) on ice for 30 min and then co-cultured with PBMC-derived M2 macrophages (prepared as described above) at a ratio of 1:2 (OVCAR8/B cells: macrophages) for 2 hours in a humidified 5% CO₂ incubator at 37° C. Phagocytic events were quantified by counting the number of red fluorescent (pHrodo+) cells per well using a Celigo Imaging Cytometer (Nexcelom). EmGFP was used for the visualization of cancer cells in imaging

Phagocytic events were also quantified using a previously-described flow cytometry-based assay (Barkal et al., Nature 2019, 572(7769):392-396; Freile et al., Biomedicines 2022, 10(5):1175). In brief, M2 macrophages co-cultured with OVCAR8/EmGFP-luc cells were detached using TrypLE Express (Gibco™, cat #12604013), stained with APC anti-human CD11b clone ICRF44 (BioLegend, Cat #982604) and analyzed by flow cytometry (BioRad ZE5). Double-positive EmGFP+CD11b+ cells were considered phagocytic events.

Animal Experiments: All mouse studies were performed in accordance with the Guide for the Care and Use of Laboratory Animals from NIH, and a protocol (202000157) approved by the UMass Chan Medical School Institutional Animal Care and Use Committee (IACUC). Female NSG mice (n=3 per group), 6-7 weeks of age, were injected intraperitoneally with 4×10⁶ OVCAR8/EmGFP-luc cells or OVCAR8-ΔGPAA1/EmGFP-luc cells. Tumors were analyzed using bioluminescence imaging beginning 7 days post-engraftment and continuing every 7 days until day 28. Mice were injected intraperitoneally with firefly D-luciferin at 140 mg/kg in PBS and images were acquired 10 min after luciferin injection using an IVIS Spectrum (Perkin Elmer). Total flux was quantified using Living Image 4.0 software (IVIS Imaging Systems).

For macrophage depletion experiments, 200 μl of clodronate liposomes (Liposoma) was administered intraperitoneally every 4 days for 16 days before tumor implantation, followed by injection of 100 μl of clodronate liposomes every 4 days. Depletion of F4/80⁻ macrophages was verified by flow cytometry analysis peritoneal lavage using an APC anti-mouse F4/80 antibody (BioLegend, Cat #123116, clone BM8, 1:50 dilution).

For bestatin treatment, 4×10⁶ OVCAR8/EmGFP-luc cells were implanted in the peritoneal cavity of mice as described above (n=5 per group), and 7 days later bestatin (100 mg/kg) was administered daily by intraperitoneal injection for 4 weeks. Tumors were analyzed by in vivo bioluminescence imaging as described above.

For the drug combination experiment, 5×10⁶ OVCAR8/EmGFP-luc cells were mixed with Matrigel (Sigma, Cat #CLS356237) 1:1 volume and were implanted subcutaneously in the right flank of NSG mice (n=x per group). When tumors reached 75 to 100 mm³, mice were treated with either vehicle control (0.9% saline), bestatin (100 mg/kg twice daily by oral gavage), docetaxel (ShelleckChem, Cat #S1148) (5 mg/kg twice a week by tail vein injection) or a combination of both bestatin and docetaxel for 28 days. Tumors were measured every week using digital calipers and tumor volumes were calculated using the formula (V=½ (Length×Width²). Differences between control and treated groups were determined using the two-way ANOVA followed by Turkey's multiple competition test. The animal weights were recorded every week using a digital weighing balance.

In vivo Phagocytosis Assay: An in vivo phagocytosis assay was performed as described previously (Barkal et al., Nature 2019, 572(7769):392-396). Mice were implanted with 4×10⁶ OVCAR8/EmGFP-luc cells as described above and 7 days later bestatin (100 mg/kg) was administered daily by intraperitoneal injection. After 10 days of bestatin treatment, peritoneal ascites was harvested. Single-cell suspensions of ascitic cells were blocked using TruStain FcX (anti-mouse CD16/32 antibody (BioLegend, Cat #101320) for 10 min at room temperature and then stained with an APC anti-mouse F4/80 antibody (BioLegend, Cat #123116, clone BM8, 1:50 dilution) for 30 min on ice in the dark. The percentage of cells undergoing phagocytosis was calculated as the percentage of F4/80+ EmGFP+ double-positive cells in the population.

Statistics: To achieve statistical significance, all qRT-PCR and flow cytometry data were collected from experiments performed in technical triplicate, and all phagocytosis and animals experiments were carried out in biological replicates; each experiment was repeated at least twice and statistically significant results were obtained in independent biological replicates. Differences between groups were assayed using an unpaired two-sample student t-test using Prism 8. Significant differences were considered when P<0.05; *P≤0.05, **P≤0.01, ***P≤0.001, ****P≤0.0001 The error bars indicate the standard deviation for the replicates.

TABLE 1
shRNA sequences

| shRNA | shRNA ID | Sequence (5′ → 3′) |
|---|---|---|
| CD24-1 | TRCN0000057677 | CCCACGCAGATTTATTCCAGT |
| | | (SEQ ID NO: 1) |
| CD24-2 | TRCN0000057677 | CCCACGCAGATTTATTCCAGT |
| | | (SEQ ID NO: 2) |
| GPAA1-1 | TRCN0000242567 | GAGGTCTACACGCAGAGTTTC |
| | | (SEQ ID NO: 3) |
| GPAA1-2 | TRCN0000242568 | GCTGACACTGCTGGCGATTTA |
| | | (SEQ ID NO: 4) |
| GPAA1-3 | TRCN0000242569 | TGAAGCCTACCACGATGTCAA |
| | | (SEQ ID NO: 5) |
| PEPT1 | TRCN0000043302 | GCTCTTGAAATTCAGCCCGAT |
| | | (SEQ ID NO: 6) |
| PEPT2 | TRCN0000005285 | CCGTTGTTTGACTTTGTCATT |
| | | (SEQ ID NO: 7) |
| PIGP-1 | TRCN0000159948 | GCCAAAGAACTTTACACCAAA |
| | | (SEQ ID NO: 8) |
| PIGP-2 | TRCN0000160209 | CAGCCTTAAGAGATATTTCTA |
| | | (SEQ ID NO: 9) |
| PIGT-1 | TRCN0000141515 | CCTGTCATTCACACAAGGCTT |
| | | (SEQ ID NO: 10) |
| PIGT-2 | TRCN0000141797 | GCAGACCCTGTCAGTTGTATT |
| | | (SEQ ID NO: 11) |
| PIGU-1 | TRCN0000129318 | CCTGCATCATCATCGTCTGTT |
| | | (SEQ ID NO: 12) |
| PIGU-2 | TRCN0000129146 | GTTTATCCAGATCGCTGTCAT |
| | | (SEQ ID NO: 13) |
| PIGK-1 | TRCN0000050118 | GCAACTGCTTAATGGCACTAA |
| | | (SEQ ID NO: 14) |
| PIGK-2 | TRCN0000050119 | GCTCAGATAATACACCAGAAA |
| | | (SEQ ID NO: 15) |

TABLE 2
sgRNA sequences

| | Forward primer sequence |
|---|---|
| Gene | (5′→3′) |
| CD24 | CGGUGCGCGGCGCGUCUAGC |
| | (SEQ ID NO: 16) |
| GPAA1 | CCGCAUCGUCCGUUCAAGCC |
| | (SEQ ID NO: 17) |

TABLE 3
qRT-PCR primer sequences

| | Forward primer | Reverse primer |
|---|---|---|
| Gene | sequence (5′→3′) | sequence (5′→3′) |
| CD24 | TTCTCCAAGCACCCA | TGGAATAAATCTGC |
| | GCA | GTGGGTA |
| | (SEQ ID NO: 18) | (SEQ ID NO: 19) |
| GPAA1 | AGGGTCCATCTCCA | GCATAAATCGCCAGC |
| | AGAGCCTGTG | AGTGTCAGC |
| | (SEQ ID NO: 20) | (SEQ ID NO: 21) |

### Example 1: CD24 is Highly Expressed in Ovarian Tumor Cells

Innate immune checkpoints are a promising new target in medical oncology (Lentz et al., Mol Cancer Ther 2021, 20(6):961-974), and several are being explored as new targets for cancer immunotherapy, including CD47-SIRPα, B2M(MHCI)-LILRB1 and PD-1/PD-L1 (which has traditionally been viewed as a T cell immune checkpoint but has more recently been shown to regulate phagocytosis) (Feng et al., Nat Rev Cancer 2020, 19(10):568-586). A recent study has identified CD24-SIGLEC10 as a dominant innate immune checkpoint in ovarian cancer, revealing it is a promising immunotherapy target (Barkal et al., Nature 2019, 572(7769):392-396). CD24 is a small glycosylphosphatidylinositol (GPI)-anchored cell surface glycoprotein that is expressed predominantly on hematopoietic cells, such as B and activated T cells (Fang et al., Cell Mol Immunol2010, 7(2):100-3), but is frequently overexpressed in human tumors (Altevogt et al., Int J Cancer 2021, 148(3):546-559) and is associated with poor clinical outcome (Kristiansen et al., Am J Pathol 2002, 161(4):1215-21). A previous study analyzing RNA-seq data from The Cancer Genome Atlas (TCGA) and Therapeutically Applicable Research to Generate Effective Treatments (TARGET) in multiple cancers found that CD24 is most highly upregulated in ovarian cancer and, furthermore, is more highly upregulated than other known innate immune checkpoints such as CD47, PD-L1 and B2M (Barkal et al., Nature 2019, 572(7769):392-396). We investigated CD24 expression in ovarian cancer at the cellular level by performing bioinformatic analysis of single-cell RNA-seq data (using the BioTuring Browser) application for analyzing single-cell datasets), which confirmed that CD24 is highly expressed in ovarian tumor cells relative to CD47, PD-L1 and B2M (FIGS. 1A-1B).

### Example 2: Identification of Factors that Promote Cell Surface Expression of CD24 in Ovarian Cancer

To identify factors required for expression of CD24, we carried out a genome-scale CRISPR/Cas9 screen. The screen was performed in OVCAR8 human ovarian cancer cells, a cisplatin-resistant HGSOC cell line (Schilder et al., Int J Cancer 1990, 5(3):416-22). We first confirmed that shRNA-mediated knockdown of CD24 (FIGS. 2A-2B) is not required for viability (FIG. 2C) or proliferation (FIG. 2D) of OVCAR8 cells.

For the screen, OVCAR8 cells stably expressing Cas9 were transduced with the human Brunello CRISPR library, which consists of ~76,000 sgRNAs targeting ~19,000 genes (4 sgRNAs per gene) (FIG. 3A). Cells were puromycin selected for 15 days, and then stained with an anti-CD24 antibody. Cells with substantially reduced expression of CD24 (CD24^{low} cells) were isolated by fluorescence-activated cell sorting (FACS), and sgRNAs that were significantly enriched in the CD24^{low} population relative to the unsorted population were identified by deep sequencing. We defined candidates as those genes with at least three significantly enriched sgRNAs, of which at least two sgRNAs were among the top 10% most significant sgRNAs. This approach identified 21 genes in addition to CD24 itself (FIG. 3B).

The top scoring factor in the primary screen was GPAA1 (glycosylphosphatidylinositol anchor attachment 1), an essential component of the multi-subunit GPI transamidase (GPIT) complex that catalyzes the attachment of a GPI lipid anchor to the C-terminus of substrate proteins, which serves as a general mechanism for linking proteins (such as CD24) to the cell surface membrane (Kinoshita, Open Biol 2020, 10(3):190290). In addition to GPAA1, the GPIT complex consists of four other subunits—PIGK, PIGS, PIGT and PIGU (phosphatidylinositol glycan anchor biosynthesis class K, S, T and U, respectively)—and to date more than 15 other proteins have been implicated in the biogenesis of GPI-anchored proteins. Notably, PIGU, as well as other proteins involved in GPI synthesis and attachment (PIGM, N, O and P) were also identified in the primary screen (FIG. 3B). Gene Ontology (GO) enrichment analysis revealed that the most significant biological processes related to the enriched sgRNAs were GPI anchor biosynthesis and GPI anchor metabolism (FIG. 3C). We validated a subset of candidates involved in GPI anchor synthesis and attachment by knocking down each gene in parental OVCAR8 cells using two independent shRNAs and monitoring expression of cell surface CD24 by flow cytometry using an anti-CD24 antibody. The results of FIG. 3D confirmed that knockdown of GPAA1, PIGP, PIGT or PIGU decreased cell surface expression of CD24. In addition to this, we also identified PIGK in our screen as a significantly enriched candidate (though not in 10% most significantly sgRNAs). We independently validated PIGK as described above. The results of FIG. 3E confirmed that knockdown of PIGK also decreased cell surface expression of CD24.

### Example 3: Validation of GPAA1 as a Factor that Promotes Cell Surface Expression of CD24 in Ovarian Cancer

It is estimated that there are approximately 150 GPI-anchored proteins in the human proteome (Kinoshita, Open Biol 2020, 10(3):190290). Proteins that are destined to become GPI-anchored harbor an N-terminal endoplasmic reticulum (ER) localization sequence and a C-terminal GPI-attachment signal peptide, and are translocated to the ER where the attachment of a presynthesized GPI lipid anchor occurs (Kinoshita, Open Biol 2020, 10(3):190290). Once the GPI anchor is attached, the protein is shuttled to the Golgi, where it undergoes fatty acid remodeling before being transported to the plasma membrane.

The GPIT complex mediates the attachment of the GPI anchor to the substrate protein in two steps: (1) cleavage of the C-terminal GPI-attachment signal peptide at the so-called omega site, resulting in a carbonyl enzyme-substrate intermediate, and (2) formation of an amide bond between the carbonyl intermediate and a phosphoethanolamine group of the GPI lipid anchor (FIG. 4A). The first reaction is catalyzed by PIGK, a cysteine protease. The second step is thought to be catalyzed by GPAA1, which harbors a putative catalytic site that is structurally similar to that of a certain class of metallopeptidases (Eisenhaber et al., Cell Cycle 2014, 13(12):1912-7); Su et al., Biol Direct 2020, 15(1):14). Thus, GPAA1 is predicted to “work in reverse” (as a metallopeptide synthase) and catalyze amide bond formation between the carbonyl intermediate at the omega site and phosphoethanolamine of GPI (Eisenhaber et al., Cell Cycle 2014, 13(12):1912-7); Su et al., Biol Direct 2020, 15(1):14).

Interestingly, GPAA1 has been reported to be overexpressed in several types of cancers (Nagpal et al., Mod Pathol 2008, (8):979-91; Zhao et al., J Biol Chem 2012, 287(30):25230-40; Wu et al., Cancer Res 2006, 66(20):9829-36; Chen et al., Genet Mol Res 2014, 13(1):499-507), and a query of the cBio Cancer Genomics Portal (Cerami et al., Cancer Discov 2012, 2(5):401-4) revealed that GPAA1 is amplified in 30% of serous ovarian cancers (SOCs) and ~25% of high grade serous ovarian cancers (HGSOCs) (FIG. 4B). Kaplan-Meier survival analysis of published gene expression datasets from HGSOC patients (Lisowska et al., Front Oncol 2014, 4:6) reveals that high expression of GPAA1 (FIG. 4C) and CD24 (FIG. 4D) is associated with reduced probability of overall survival.

We carried out several experiments to confirm that GPAA1 is required for cell surface expression of CD24. Consistent with the results of FIG. 3D, knockdown of GPAA1 in OVCAR8 cells using one of three independent shRNAs (FIG. 4E) significantly reduced cell surface expression of CD24 as measured by flow cytometry analysis (FIG. 4F). However, shRNA-mediated knockdown of GPAA1 did not affect CD24 mRNA levels (FIG. 4G), consistent with role for GPAA1 in post-translational modification of CD24. To confirm and extend these results, we derived an OVCAR8 GPAA1 knockout (KO) cell line by CRISPR/Cas9-mediated gene editing (FIG. 4H). As a control, we also derived an OVCAR8 CD24 KO cell line. As expected, GPAA1 KO OVCAR8 cells had completely abrogated cell surface expression of CD24, comparable to that observed in CD24 KO cells, as evidenced by flow cytometry (FIG. 4I) and immunocytochemistry (FIG. 4J) using an anti-CD24 antibody. We examined whether GPAA1 KO affected the expression of non-GPI-linked cell surface proteins involved in immunity. The flow cytometry analysis showed that GPAA1 knockout did not alter the cell surface expression of non-GPI linked proteins, CD47, MHC-1 and B2M respectively (FIGS. 4K-4M). Thus, as expected, the effects of GPAA1 KO are specific to GPI-linked cell surface proteins.

### Example 4: Genetic Knockout of GPAA1 Promote Phagocytosis of Ovarian Cancer Cells by Human PBMC-Derived Macrophage

As stated above, CD24 promotes immune evasion by providing an anti-phagocytosis (“don't eat me”) signal that prevents targeting and engulfment by macrophages. Therefore, bestatin-mediated reduction in CD24 cell surface expression is expected to enhance phagocytosis of ovarian cancer cells by macrophages. To test this possibility, we performed phagocytosis assays (Barkal et al., Nature 2019, 572(7769):392-396; Bachynski et al., J Pediatr Ophtalmol Strabismus 1989, 26(2):56-60). First, we performed a fluorescence-based phagocytosis assay in which cells are coated with a pH-sensitive fluorescent dye called pHrodo, which has a low fluorescence intensity at neutral pH (outside the cell) but fluoresces brightly at acidic pH (such as phagocytic lysosomes), allowing phagocytic events to be detected by microscopy (FIG. 5A) (Miksa et al., J Immunol Methods 2009, 342(1-2):71-7; Lindner et al., Biotechniques 2020, 68(5):245-250). To facilitate quantification of phagocytic events, we adapted the assay for use with a Celigo imaging cytometer. Parental or GPAA1 KO or CD24 KO OVCAR8 cells stably expressing green fluorescent protein (GFP) were stained with pHrodo, and then overlaid onto macrophages derived from human peripheral blood mononuclear cells (PBMCs) that had been treated with monocyte attachment medium (MAM) for 1 hour followed by macrophage colony-stimulating factor (M-CSF) and interleukins IL-4 and IL-13 for 7 days to induce differentiation and polarization to M2 cells to mimic the TAM phenotype (FIG. 5B). In another phagocytosis assay, GFP-expressing parental or GPAA1 KO or CD24 KO OVCAR8 cells were co-cultured with PBMC-derived M2 macrophages, and 3 hours later flow cytometry was performed to detect cells that were double-positive for GFP and CD11b (a macrophage marker, a surface receptor on macrophages that plays a critical role in phagocytosis), indicative of macrophage engulfment of a cancer cell. The flow cytometry results showed double-positive macrophages (GFP+ and CD11b+) in parental, GPAA1 KO, and CD24 KO OVCAR8 cells (FIG. 5C). The results showed that parental OVCAR8 cells had a relatively low phagocytic index (e.g., number of GFP+CD11b+ cells), which was substantially increased by KO of CD24, as expected. Similarly, the phagocytic index was increased by KO of GPAA1.

### Example 5: The Metallo-Aminopeptidase Inhibitor Bestatin Reduces Cell Surface Expression of CD24 in Ovarian Cancer Cells

GPAA1 is a transmembrane protein with a luminal domain that has a sequence and predicted structure that is similar to that of M28-type peptidases (Eisenhaber et al., Cell Cycle 2014 13(12):1912-7; Su et al., Biol Direct 2020, 15(1):14), a family of metallopeptidases that carry one or more co-catalytic metal ions (most often zinc) in a tetrahedral coordination (ncbi.nlm.nih.gov/Structure/cdd/c114876). Metallopeptidases are enzymes that catalyze the cleavage of amino acids from either the amino terminus (aminopeptidases) or carboxy terminus (carboxypeptidases) of proteins or peptides and have emerged as novel targets for the treatment of a variety of human diseases (Mucha et al., Biochimie 2010, 92(11):1509-29; Arolas et al., Curr Pharm Des 2007, 13(4):349-66). Several small molecule metallo-aminopeptidase inhibitors are being developed for potential clinical use (Gonzalez-Bacerio et al., Microbial Cell 2021, 8(10):239-246). In particular, inhibitors of the aminopeptidase leukotriene A4 hydrolase, a rate limiting enzyme required for production of the pro-inflammatory mediator leukotriene B4, are in development for the treatment of inflammatory conditions such as lymphedema and hypertension (Penning, Curr Pharm Des 2001, 7(3):163-79). Of these inhibitors, bestatin (Ubenimex), a potent inhibitor of multiple metallo-aminopeptidases (Scornik and Botbot, Curr Drug Metab 2001, 2(1):67-85), is the most clinically advanced and has received orphan drug designation from the FDA for treatment of pulmonary arterial hypertension. Bestatin has also been shown to have immunostimulatory and antitumor activity (Talmadge et al., Cancer Res 1986, 46(9):4505-10; Ichinose et al., J Natl Cancer Inst 2003, 95(8):605-10), and has been approved in Japan as a chemotherapy adjuvant for acute non-lymphocytic leukemia. The rationale for the use of aminopeptidase inhibitors as anti-cancer agents is that they can induce amino acid deprivation, which is deleterious to rapidly growing cells. Bestatin is a natural compound (FIG. 6A) isolated from Streptomyces oliverticuli, and is an orally active, well-characterized, safe, and well-tolerated drug.

Due to the predicted structural similarity between GPAA1 and M28-type metallopeptidases, we hypothesized that GPAA1 may be inhibited by bestatin and carried out a series of experiments to test this possibility. We first performed a cellular thermal stability shift assay (CETSA), a method for evaluating drug-target interactions in cells (Jafari et al., Nature Protoc 2014, (9):2100-22). In a CETSA, cells are treated with the compound of interest, and then heated to denature and precipitate proteins. Cells are then lysed, and cell debris and aggregates are separated from the soluble protein fraction by centrifugation. The assay relies on the principle of thermodynamic stabilization of a protein as a result of ligand binding; unbound proteins denature and precipitate at elevated temperatures, whereas ligand-bound proteins remain in solution. The ligand-bound proteins in the soluble fraction are analyzed by immunoblotting to quantify the changes in thermal stability. FIG. 6B shows that the thermal stability of GPAA1 was increased in the presence of bestatin relative to DMSO, suggesting bestatin binds to GPAA1.

We next sought to determine whether bestatin, like GPAA1 knockdown or knockout, would reduce cell surface expression of CD24. The flow cytometry analysis of FIG. 6C shows that treatment of OVCAR8 cells with bestatin reduced CD24 cell surface expression in a dose-dependent manner. Similar results were obtained in another human ovarian cancer cell line, SKOV3 (FIG. 6D). Notably, bestatin had no significant effect on total CD24 levels, as assessed following permeabilization of cells (FIG. 6E), indicating that bestatin specifically decreases cell surface expression of CD24. Also, bestatin had no effect on cell viability of OVCAR8 or SKOV3 cells (FIG. 6F). Since bestatin reduced CD24 expression, it is possible that bestatin treatment would, like GPAA1 KO, enhance macrophage-mediated phagocytosis of ovarian cancer cells. Thus, FIGS. 6G-6I show that treatment of OVCAR8 and SKOV3 cells with bestatin substantially increased phagocytosis.

### Example 6: Sensitivity of Ovarian Cancer Cells to Bestatin Requires Expression of the Dipeptide Transporter PEPT1 or PEPT2

Bestatin is a dipeptide-mimetic drug (FIG. 6A) that is transported into the cell via a dipeptide transporter, either PEPT1 (also called SLC15A1) or PEPT2 (also called SLC15A2) (Saito and Inui, Am J Physiol 1993, 265(2 Pt 1):G289-94). Interestingly, TCGA data indicate that PEPT1 and PEPT2 are overexpressed in ovarian cancer compared to normal ovarian tissue (FIG. 7A). In normal tissues in the body, PEPT1 is exclusively expressed in intestinal tissue, whereas PEPT2 is expressed more broadly and at low levels (Human Protein Atlas, data not shown). Consistent with this observation, we detected expression of PEPT1 and PEPT2 in multiple human ovarian cancer cell lines including OVCAR8, SKOV3, OVCAR4, NCI-ADDRES and IGROV1 (FIG. 7B). Notably, the ability of bestatin to decrease CD24 cell surface expression was abrogated by knockdown of PEPT1 or PEPT2 (FIG. 7C). Previous studies have shown that MCF7 breast cancer cells, which express CD24 (Barkal et al., Nature 2019, 572(7769):392-396), express PEPT1 and PEPT2 at low or undetectable levels (Human Protein Atlas, data not shown). Consistent with these previous results, we also found that in MCF7 cells PEPT1 and PEPT2 protein levels were not detectable (FIG. 7B). Notably, treatment of MCF7 cells with bestatin did not decrease CD24 cell surface expression (FIG. 7D), whereas shRNA-mediated knockdown of GPAA1 reduced CD24 cell surface expression (FIG. 7E). Collectively, these results suggest that ovarian cancer cells are selectively sensitive to bestatin due to expression of PEPT1 and/or PEPT2, which mediate drug uptake.

### Example 7: Other Metallo-Aminopeptidase Inhibitors Decrease CD24 Cell Surface Expression and Increase Phagocytosis of Ovarian Cancer Cells

We next investigated whether other small molecule aminopeptidase inhibitors would, like bestatin, reduce cell surface expression of CD24 and induce macrophage-mediated phagocytosis of ovarian cancer cells. As a first test of this possibility, we tested two other aminopeptidase inhibitors. Tosedostat (also called CHR-2797) (Jenkins et al., Leuk Res 2011, 35(5):677-81) is another clinically advanced metallo-aminopeptidase inhibitor that has been tested in a number of phase 1/2 clinical trials, alone and in combination with other anti-cancer agents, for treatment of patients with acute myeloid leukemia (AML), multiple myeloma, myeloid dysplastic syndrome (MDS) (NCT00780598; NCT00689000; NCT02452346; NCT01567059) and advanced solid tumors (NCT00737555; NCT00692354). Like bestatin, tosedostat is a dipeptide mimetic (FIG. 8A). FIGS. 8B-8E demonstrate that treatment of OVCAR8 or SKOV3 cells with tosedostat reduced CD24 cell surface expression and increased phagocytosis. Another metallo-aminopeptidase inhibitor, ARM1 (also called 4BSA) (FIG. 8F) (Stsiapanava et al., Proc Natl Acad Sci USA 2014, 111(11):4227-32), also decreased CD24 cell surface expression and increased phagocytosis of OVCAR8 cells (FIGS. 8G-8H).

### Example 8: Unlike an Anti-CD24 Monoclonal Antibody, Bestatin and Tosedostat Do Not Induce Phagocytosis of Normal B Cells

Monoclonal antibodies targeting cell surface proteins may act, in part, through opsonization, an immune process that marks cells for phagocytosis by Fc-receptor-expressing immune cells in the tumor microenvironment (Tay et al., Front Immunol 2019, 10:332). Indeed, the pro-phagocytic activity of anti-CD24 monoclonal antibodies has been shown to stem not only from CD24/SIGLEC10 checkpoint inhibition but also from opsonization and induction of antibody-dependent cellular phagocytosis (Alvarez Freile et al., Biomedicines 2022, 10(5):1175). Thus, small molecules such as bestatin and tosedostat, which act by reducing CD24 levels, may provide a significant therapeutic advantage over anti-CD24 antibodies, which may induce unwanted phagocytosis of normal CD24-expressing cells. To test this possibility, we compared the ability of bestatin or tosedostat and an anti-CD24 antibody to induce phagocytosis of normal B cells. The flow cytometry analysis of FIG. 9A shows that B cells express high levels of CD24, which were not diminished by treatment with bestatin or tosedostat. Consistent with this observation, B cells did not undergo phagocytosis upon treatment with bestatin or tosedostat but, as expected, underwent phagocytosis upon treatment with an anti-CD24 antibody (FIG. 9B). By contrast, as expected, OVCAR8 cells were susceptible to bestatin, tosedostat and anti-CD24 antibody treatment (FIG. 9C). These results suggest that, unlike anti-CD24 antibodies, bestatin and tosedostat have selectivity toward CD24+ ovarian cancer cells relative to normal CD24+ B cells.

### Example 9: Genetic or Pharmacological Inhibition of GPAA1 Suppresses Growth of Tumors Derived from Human Ovarian Cancer Cells in Xenograft Mice

We asked whether genetic knockout or pharmacological inhibition of GPAA1 would reduce ovarian cancer tumor growth in mice. In the first experiment, female NOD scid gamma (NSG) mice 6-8 weeks old were implanted by intraperitoneal (IP) injection with 4×10⁶ OVCAR8 cells stably expressing GFP and luciferase (OVCAR8-GFP-Luc cells) or OVCAR8-GFP-Luc GPAA1 KO cells, and tumor growth was measured weekly by in vivo bioluminescence imaging (BLI). The results of FIG. 10A show that OVCAR8-GFP-Luc GPAA1 KO cells had reduced tumor growth compared to parental OVCAR8-GFP-Luc cells. To test whether the reduced tumor growth of OVCAR8-GFP-Luc GPAA1 KO cells was macrophage dependent, we injected mice with liposomes containing clodronate, an agent that depletes macrophages (Weisser et al., J Vis Exp 2012, (66):4105). FIG. 10B shows that the ability of GPAA1 KO cells to form tumors was restored by clodronate-mediated macrophage depletion.

We next tested whether bestatin treatment would also reduce ovarian cancer tumor growth. As above, 4×10⁶ parental OVCAR8-GFP-Luc cells were implanted by IP injection and 1 week later vehicle or bestatin (100 mg/kg/d) was administered by IP injection, and tumor growth was monitored weekly by BLI. FIG. 10C shows that bestatin significantly reduced tumor growth. A hallmark of ovarian cancer that is also observed in mouse models is the build-up of fluid (ascites) in the peritoneal cavity (Ahmed and Stenvers, Front Oncol 2013, 3:256). FIG. 10D shows that bestatin-treated mice had significantly reduced ascites volume compared to vehicle-treated mice. Finally, we performed an in vivo phagocytosis assay on the aspirated ascites fluid (Barkal et al., Nature 2019, 572(7769):392-396). In brief, ascites fluid, which contains OVCAR8-GFP-Luc cells, was stained for the mouse F4/80 antigen (a murine macrophage marker) and analyzed by flow cytometry for GFP+F4/80+ double-positive cells, indicative of macrophage-engulfed ovarian cancer cells. FIG. 10E shows that bestatin treatment resulted in an increase in GFP+F4/80+ double-positive cells. Representative confocal microscopy images of cancer cells engulfed by macrophages isolated from the ascites fluid are shown (FIG. 10E).

### Example 10: Docetaxel Enhances the Ability of Bestatin to Suppress Growth of Ovarian Tumor Xenografts

Previous studies have demonstrated that chemotherapeutic drugs, including doxorubicin, carboplatin, and docetaxel, can trigger immunological cell death, a form of cell death that is characterized by the release of death-associated molecular signals (DAMPs), such as ATP and HMGB1, and translocation of the pro-phagocytic signal CALR to the cell surface, which may induce phagocytosis of cancer cells (Krysko et al., Nat Rev Cancer 2012 12(12):860-75; Hodge et al., Int J Cancer 2013, 133(3):624-36). In particular, docetaxel has been shown to induce CALR surface translocation (Hodge et al., Int J Cancer 2013, 133(3):624-36). Consistent with these previous results, we found that docetaxel induced robust CALR cell surface expression in OVCAR8 cells, whereas carboplatin and doxorubicin did not (FIG. 11A). Moreover, combined treatment of docetaxel and bestatin significantly increased macrophage-mediated phagocytosis of OVCAR8 cells compared to single treatment alone (FIG. 11B). Further experiments were conducted to investigate the effects of single or combinatorial treatment of these drugs on tumor growth in vivo. NSG mice were subcutaneously xenografted with 5×10⁶ OVCAR8 cells and when tumors reached 75-100 mm³ mice were treated with bestatin (by oral gavage), docetaxel (by tail vein injection) or both, and tumor growth was monitored. The results showed that combinatorial treatment led to a significant reduction in tumor growth (FIG. 11C) without significantly affecting the health of the mice, as evidenced by the absence of weight loss in drug-treated mice (FIG. 11D).

### Example 11: Suppression of Ovarian Tumor Growth by Bestatin and Other Aminopeptidase Inhibitors

Our model for how bestatin and other small molecule aminopeptidase inhibitors reduce ovarian cancer growth is shown in FIG. 12. Aminopeptidase inhibitors block the peptide synthetase activity of GPAA1, thereby inhibiting its ability to add a GPI lipid anchor to CD24, resulting in reduced cell surface expression of CD24. In the absence of the CD24 “don't eat me” signal, macrophages are able to phagocytose and kill ovarian cancer cells, resulting in reduced tumor growth.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating ovarian cancer, the method comprising administering to a subject in need thereof an effective amount of an inhibitor of a glycosylphosphatidylinositol (GPI) pathway protein.

2. The method of claim 1, wherein the GPI pathway protein comprises glycosylphosphatidylinositol anchor attachment 1 (GPAA1), phosphatidylinositol glycan anchor biosynthesis class P (PIGK), phosphatidylinositol glycan anchor biosynthesis class P (PIGP), phosphatidylinositol glycan anchor biosynthesis class T (PIGT), or phosphatidylinositol glycan anchor biosynthesis class U protein (PIGU).

3. The method of claim 1, wherein the inhibitor comprises a small molecule inhibitor, a peptide inhibitor, an antibody or antigen binding fragment thereof, an agent that inhibits expression of the GPI pathway protein, or a combination thereof.

4. The method of claim 3, wherein the small molecule inhibitor comprises a metallo-aminopeptidase inhibitor.

5. The method of claim 4, wherein the metallo-aminopeptidase inhibitor comprises bestatin, LYP, LYP3, tosedostat, ARM1, or a combination thereof.

6. The method of claim 3, wherein the agent that inhibits expression of the GPI pathway protein comprises a short interfering nucleic acid (siNA), a short interfering RNA (siRNA), a double-stranded RNA (dsRNA), a micro-RNA (miRNA), a short hairpin RNA (shRNA), or a combination thereof.

7. The method of claim 1, wherein the inhibitor is formulated in a pharmaceutical composition, which further comprises a pharmaceutically acceptable carrier.

8. The method of claim 1, wherein the subject is a human subject having or at risk for having ovarian cancer.

9. The method of claim 1, wherein the ovarian cancer comprises epithelial ovarian cancer, germ cell ovarian cancer, stromal cell ovarian cancer, small cell carcinoma (SCCO), or combinations thereof.

10. The method of claim 1, wherein the ovarian cancer comprises chemo-resistant ovarian cancer.

11. The method of claim 1, wherein ovarian cancer cells in a biological sample obtained from the subject express CD24.

12. The method of claim 1, wherein ovarian cancer cells in a biological sample obtained from the subject express GPAA1.

13. The method of claim 1, wherein ovarian cancer cells in a biological sample obtained from the subject express PEPT1 and/or PEPT2.

14. The method of claim 1, further comprising administering to the subject an additional anti-cancer therapy.


SEQUENCE LISTING (21 sequences)

SEQ ID NO: 1 - RNA, 21 nt, synthetic construct
  source 1..21: mol_type = other RNA
cccacgcagatttattccagt

SEQ ID NO: 2 - RNA, 21 nt, synthetic construct
  source 1..21: mol_type = other RNA
cccacgcagatttattccagt

SEQ ID NO: 3 - RNA, 21 nt, synthetic construct
  source 1..21: mol_type = other RNA
gaggtctacacgcagagtttc

SEQ ID NO: 4 - RNA, 21 nt, synthetic construct
  source 1..21: mol_type = other RNA
gctgacactgctggcgattta

SEQ ID NO: 5 - RNA, 21 nt, synthetic construct
  source 1..21: mol_type = other RNA
tgaagcctaccacgatgtcaa

SEQ ID NO: 6 - RNA, 21 nt, synthetic construct
  source 1..21: mol_type = other RNA
gctcttgaaattcagcccgat

SEQ ID NO: 7 - RNA, 21 nt, synthetic construct
  source 1..21: mol_type = other RNA
ccgttgtttgactttgtcatt

SEQ ID NO: 8 - RNA, 21 nt, synthetic construct
  source 1..21: mol_type = other RNA
gccaaagaactttacaccaaa

SEQ ID NO: 9 - RNA, 21 nt, synthetic construct
  source 1..21: mol_type = other RNA
cagccttaagagatatttcta

SEQ ID NO: 10 - RNA, 21 nt, synthetic construct
  source 1..21: mol_type = other RNA
cctgtcattcacacaaggctt

SEQ ID NO: 11 - RNA, 21 nt, synthetic construct
  source 1..21: mol_type = other RNA
gcagaccctgtcagttgtatt

SEQ ID NO: 12 - RNA, 21 nt, synthetic construct
  source 1..21: mol_type = other RNA
cctgcatcatcatcgtctgtt

SEQ ID NO: 13 - RNA, 21 nt, synthetic construct
  source 1..21: mol_type = other RNA
gtttatccagatcgctgtcat

SEQ ID NO: 14 - RNA, 21 nt, synthetic construct
  source 1..21: mol_type = other RNA
gcaactgcttaatggcactaa

SEQ ID NO: 15 - RNA, 21 nt, synthetic construct
  source 1..21: mol_type = other RNA
gctcagataatacaccagaaa

SEQ ID NO: 16 - RNA, 20 nt, synthetic construct
  source 1..20: mol_type = other RNA
cggtgcgcggcgcgtctagc

SEQ ID NO: 17 - RNA, 20 nt, synthetic construct
  source 1..20: mol_type = other RNA
ccgcatcgtccgttcaagcc

SEQ ID NO: 18 - DNA, 18 bp, synthetic construct
  source 1..18: mol_type = other DNA
ttctccaagcacccagca

SEQ ID NO: 19 - DNA, 21 bp, synthetic construct
  source 1..21: mol_type = other DNA
tggaataaatctgcgtgggta

SEQ ID NO: 20 - DNA, 24 bp, synthetic construct
  source 1..24: mol_type = other DNA
agggtccatctccaagagcctgtg

SEQ ID NO: 21 - DNA, 24 bp, synthetic construct
  source 1..24: mol_type = other DNA
gcataaatcgccagcagtgtcagc